(19) United States
(12) Patent Application Publication
(10) Pub. No.: US 2023/0416207 A1
(43) Pub. Date: Dec. 28, 2023

(54) CRYSTALLINE FORMS OF QUINAZOLINONE COMPOUND AND PROCESS FOR PREPARING THE SAME

(71) Applicant: CRYSTAL PHARMATECH CO., LTD., Suzhou, Jiangsu (CN)

(72) Inventors: Xiaoyu ZHANG, Suzhou (CN); Zhixiong CHEN, Suzhou (CN); Xia LU, Suzhou (CN)

(73) Assignee: CRYSTAL PHARMATECH CO., LTD., Suzhou, Jiangsu (CN)

(21) Appl. No.: 18/253,387

(22) PCT Filed: Nov. 17, 2021
(86) PCT No.: PCT/CN2021/131203

§ 371 Date: May 17, 2023

(30) Foreign Application Priority Data
Nov. 17, 2020 (CN) 202011289751.1

(51) Int. Cl. C07D 239/91

(52) U.S. Cl. CPC C07D 239/91; C07B 2200/13

(57) ABSTRACT

Provided are a crystal form D, a crystal form AJ, a crystal form AL, a crystal form AZ, a crystal form AF, a crystal form Z and a crystal form AE of a compound of formula (I), a process for preparing the same and the use thereof. The crystal forms have advantages in at least one aspect of solubility, melting point, stability, dissolution, hygroscopicity, adhesiveness, fluidity, biological effectiveness, processability, purification effect, preparation production, safety, etc., thus providing a new better choice for the preparation of pharmaceutical preparations containing the compound of formula (I), and having important significance for drug development.

### CROSS-REFERENCE TO RELATED APPLICATIONS

This application claims the priory of Chinese Patent Application No. 202011289751.1 filed on Nov. 17, 2020, the contents of which are hereby incorporated by reference.

### TECHNICAL FIELD

The present disclosure relates to the field of pharmaceutical chemistry, and in particular to crystalline forms of a quinazolinone compound and process for preparing the same.

### BACKGROUND

Transient receptor potential vanilloid 1 (TRPV1) (also known as VR-1, vanilloid receptor or capsaicin receptor) is a non-selective cation channel, which is mainly distributed in nociceptive neurons. TRPV1 plays an important role in mediating inflammatory pain, visceral pain, cancer pain and even nociceptive sensitization. Also, it is involved in inflammation, immune activation and airway hyperreactive stress process. 4-(7-hydroxy-4-oxo-2-propan-2-ylquinazolin-3-yl) benzonitrile is an oral and highly efficient TRPV1 inhibitor. Its structural formula is as follows:

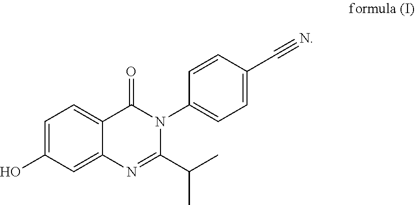 [SMILES: CC(C)c1nc2cc(O)ccc2c(=O)n1-c1ccc(C#N)cc1]

The inhibitor shown in formula (I) could ease pain by blocking the TRPV1 activation effects of capsaicin, proton and heat, and blocking the conduction of pain signals. Accordingly, the inhibitor shown in formula (I) could be used for the treatment or prevention of chronic or acute pain. Also, it could be used as anti-inflammatory agents, anti-edema agents and antianaphylaxis agents, such as postoperative pain, gout, cancer pain, psoriasis and asthma. Clinical trials have shown that the inhibitor shown in formula (I) has certain therapeutic potential in postoperative pain and neurogenic bladder dysfunction.

WO2005120510A1 discloses formula (I) and process for its preparation. Patent WO2010084050A2 discloses one crystalline form of formula (I), namely Form B, obtained by complicated process for preparation and washing. WO2020165840A1 discloses eight crystalline forms of formula (I), namely Forms A, C, E, F, G, J, K, and L. So far, no other crystalline forms are disclosed.

In addition, different crystalline forms of the same drug may have different physical and chemical properties, such as solubility, dissolving rate, melting point and stability, thus affecting its working effect in human body. Therefore, it is necessary to carry out a comprehensive and systematic polymorph screening, and obtain crystalline forms with good solubility and high stability to provide more and better choices for the follow-up pharmaceutical development.

### SUMMARY

The present disclosure provides seven crystalline forms, i.e., Forms D, AJ, AL, AZ, AF, Z, and AE, and processes for preparation thereof.
1. D-type crystal of a compound represented by formula (I) 4-(7-hydroxy-4-oxo-2-propan-2-ylquinazolin-3-yl) benzonitrile, i.e., crystalline Form D, which is characterized in that, by using Cu-Kα radiation, crystalline Form D has an X-ray powder diffraction pattern comprising characteristic peaks at the 2θ values of 6.7°±0.2°, 10.5°±0.2°, 4.2°±0.2°,

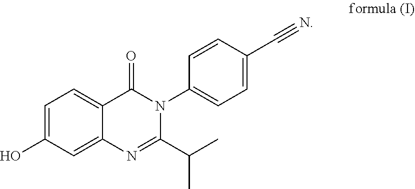 [SMILES: CC(C)c1nc2cc(O)ccc2c(=O)n1-c1ccc(C#N)cc1]

2. The process for preparing Form D according to the above-mentioned item 1, including:
(1) dissolving compound of formula (I) into a solvent which includes a mixture of an alcohol solvent and pure water, and then evaporate the solution until precipitation to obtain Form D; or
(2) dissolving compound of formula (I) into an alcohol solvent, adding a polymer in 0.5%-15% mass of compound of formula (I), the polymer including polyvinyl pyrrolidone (PVP), polyvinyl alcohol (PVA), polyvinylchloride (PVC), polyvinyl acetate (PVAC), hypromellose (HPMC), methyl cellulose (MC), polycaprolactone (PCL), polyethylene glycol (PEG), poly (methyl methacrylate) (PMMA), sodium alginate (SA) or hydroxyethyl cellulose (HEC); evaporating the solution with induction of the polymer until precipitation to obtain Form D.
3. AL-type crystal of a compound represented by formula (I) 4-(7-hydroxy-4-oxo-2-propan-2-ylquinazolin-3-yl) benzonitrile, i.e., crystalline Form AL, which is characterized in that, by using Cu-Kα radiation, crystalline Form AL has an X-ray powder diffraction pattern comprising characteristic peaks at the 2θ values of 7.4°±0.2°, 14.9°±0.2°, 12.0°±0.2°,

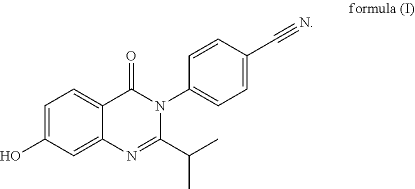 [SMILES: CC(C)c1nc2cc(O)ccc2c(=O)n1-c1ccc(C#N)cc1]

4. The process for preparing Form AL according to the above-mentioned item 3, including:
(1) dissolving compound formula (I) in ester solvent, and then evaporating the solution until precipitation, heating the obtained solid at 264° C. to obtain Form AL; or
(2) dissolving compound formula (I) into cyclic ether solvent, and then evaporating the solution until precipitation to obtain Form AL.
5. Z-type crystal of a compound represented by formula (I) 4-(7-hydroxy-4-oxo-2-propan-2-ylquinazolin-3-yl) benzonitrile, i.e., crystalline Form Z, which is characterized in that, by using Cu-Kα radiation, crystalline Form Z has an X-ray powder diffraction pattern comprising characteristic peaks at the 2θ values of 11.1°±0.2°, 18.5°±0.2°, 20.1°±0.2°,

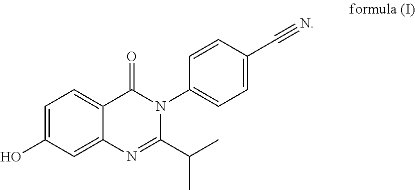 [SMILES: CC(C)c1nc2cc(O)ccc2c(=O)n1-c1ccc(C#N)cc1]

6. The process for preparing Form Z according to the above-mentioned item 5, including:
(1) dissolving compound formula (I) in ester solvent, and then evaporating the solution to obtain Form Z; or
(2) dissolving compound formula (I) in ketone, ester or cyclic ether solvent, and then evaporating the solution at high temperature until precipitation to obtain Form Z; or
(3) dissolving compound formula (I) in alcohol solvent, and then dropwise adding alkane solvent into the solution until precipitation to obtain Form Z.
7. AJ-type crystal of a compound represented by formula (I) 4-(7-hydroxy-4-oxo-2-propan-2-ylquinazolin-3-yl) benzonitrile, i.e., crystalline Form AJ, which is characterized in that, by using Cu-Kα radiation, crystalline Form AJ has an X-ray powder diffraction pattern comprising characteristic peaks at the 2θ values of 24.5°±0.2°, 14.0°±0.2°, 10.5°±0.2°,

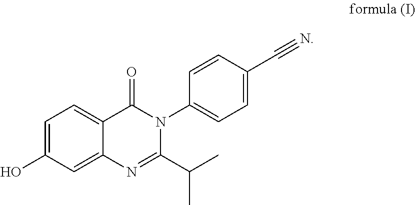 [SMILES: CC(C)c1nc2cc(O)ccc2c(=O)n1-c1ccc(C#N)cc1]

8. The process for preparing Form AJ according to the above-mentioned item 7, including:
dissolving compound formula (I) in alcohol, ketone or cyclic ether solvent, and then dropwise adding pure water or alkane solvent into the solution until precipitation; drying the solid at room temperature, and then heating the obtained solid to 200° C.˜261° C. under N₂ flow to obtain Form AJ.
9. AZ-type crystal of a compound represented by formula (I) 4-(7-hydroxy-4-oxo-2-propan-2-ylquinazolin-3-yl) benzonitrile, i.e., crystalline Form AZ, which is characterized in that, by using Cu-Kα radiation, crystalline Form AZ has an X-ray powder diffraction pattern comprising characteristic peaks at the 2θ values of 7.3°±0.2°, 7.9°±0.2°, 17.2°±0.2°,

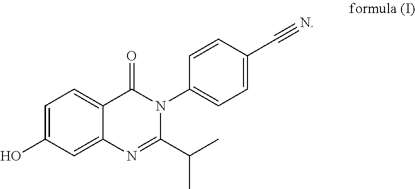 [SMILES: CC(C)c1nc2cc(O)ccc2c(=O)n1-c1ccc(C#N)cc1]

10. The process for preparing Form AZ according to the above-mentioned item 9, including:
dissolving compound formula (I) in ester solvent, and then dropwise adding alkane solvent into the solution until precipitation; drying the solid at room temperature, and then keeping the obtained solid for about 2 months to obtain Form AZ.
11. AF-type crystal of a compound represented by formula (I) 4-(7-hydroxy-4-oxo-2-propan-2-ylquinazolin-3-yl) benzonitrile, i.e., crystalline Form AF, which is characterized in that, by using Cu-Kα radiation, crystalline Form AF has an X-ray powder diffraction pattern comprising characteristic peaks at the 2θ values of 8.6°±0.2°, 7.6°±0.2°,

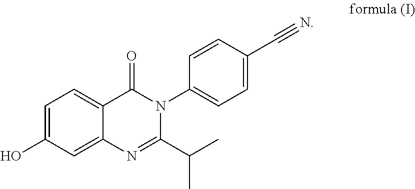 [SMILES: CC(C)c1nc2cc(O)ccc2c(=O)n1-c1ccc(C#N)cc1]

12. The process for preparing Form AF according to the above-mentioned item 11, including:
dissolving compound formula (I) in ketone solvent, and then evaporating the solution until precipitation; heating the obtained solid at 50° C.˜75° C., and then cooling back to room temperature to obtain Form AF.
13. AE-type crystal of a compound represented by formula (I) 4-(7-hydroxy-4-oxo-2-propan-2-ylquinazolin-3-yl) benzonitrile, i.e., crystalline Form AE, which is characterized in that, by using Cu-Kα radiation, crystalline Form AE has an X-ray powder diffraction pattern comprising characteristic peaks at the 2θ values of 12.6°±0.2°, 4.9°±0.2°, 14.0°±0.2°,

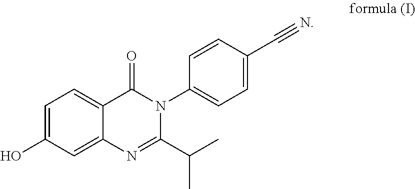 [SMILES: CC(C)c1nc2cc(O)ccc2c(=O)n1-c1ccc(C#N)cc1]

14. The process for preparing Form AE according to the above-mentioned item 13, including:
dissolving compound formula (I) in ester or ether solvent, and then evaporating the solution until precipitation to obtain Form AE.
15. A pharmaceutical composition, containing the crystal according to any one of the above-mentioned items 1, 3, 5, 7, 9, 11, 13 and a pharmaceutically acceptable carrier.
16. A pharmaceutical composition having a TRPV1 inhibitory activity, containing the crystal according to any one of the above-mentioned items 1, 3, 5, 7, 9, 11, 13 as an active component.
17. A prophylactic or curative drug for postoperative pain, neurogenic bladder dysfunction, cancer pain, myofascial pain, inflammatory diseases, psoriasis, eczema, asthma, and pneumoconiosis, which contains the crystal according to any one of the above-mentioned items 1, 3, 5, 7, 9, 11, 13 as an active component.

Compared with the prior art, crystalline forms D, AJ, AL, AZ, AF, Z and AE of the compound of formula (I) provided by the present disclosure have the advantages in at least one of solubility, melting point, stability, dissolution, hygroscopicity, adhesion, fluidity, biological effectiveness, processing performance, purification, formulation production, safety, and the like, which provides a new and better choice for the preparation of pharmaceutical formulations of the TRPV1 inhibitor, and has a very important significance for the drug development.

### BRIEF DESCRIPTION OF THE DRAWINGS

FIG. 1 illustrates an X-ray powder diffraction (XRPD) pattern of Form D.

FIG. 2 illustrates an XRPD pattern of Form AJ.

FIG. 3 illustrates an XRPD pattern of Form AL.

FIG. 4 illustrates an XRPD pattern of Form AZ.

FIG. 5 illustrates an XRPD pattern of Form AF.

FIG. 6 illustrates an XRPD pattern of Form Z.

FIG. 7 illustrates an XRPD pattern of Form AE.

FIG. 8 illustrates a TGA curve of Form D.

FIG. 9 illustrates a DSC curve of Form D.

FIG. 10 illustrates ¹H NMR spectrum of Form D.

FIG. 11 illustrates a TGA curve of Form AZ.

FIG. 12 illustrates a DSC curve of Form AZ.

FIG. 13 illustrates ¹H NMR spectrum of Form AZ.

FIG. 14 illustrates a TGA curve of Form AF.

FIG. 15 illustrates a DSC curve of Form AF.

FIG. 16 illustrates a TGA curve of Form Z.

FIG. 17 illustrates a DSC curve of Form Z.

FIG. 18 illustrates a TGA curve of Form AE.

FIG. 19 illustrates a DSC curve of Form AE.

FIG. 20 illustrates ¹H NMR spectrum of Form AE.

FIG. 21 illustrates a TGA curve of Form AL.

FIG. 22 illustrates a DSC curve of Form AL.

FIG. 23 illustrates ¹H NMR spectrum of Form AL.

FIG. 24 illustrates solubility profile of different crystal forms in SGF.

FIG. 25 illustrates solubility profile of different crystal forms in FaSSIF.

FIG. 26 illustrates XRPD overlay of Form D under 25° C./60% RH and 40° C./75% RH.

FIG. 27 illustrates XRPD overlay of Form AL under 25° C./60% RH and 40° C./75% RH.

FIG. 28 illustrates XRPD overlay of Form Z under 25° C./60% RH and 40° C./75% RH.

FIG. 29 illustrates DVS plot of Form B.

FIG. 30 illustrates XRPD overlay of Form B before and after DVS test.

FIG. 31 illustrates DVS plot of Form Z.

FIG. 32 illustrates XRPD overlay of Form Z before and after DVS test.

FIG. 33 illustrates PLM image of Form Z.

FIG. 34 illustrates PLM image of Form AL.

FIG. 35 illustrates a particle size distribution (PSD) diagram of Form D.

FIG. 36 illustrates a PSD diagram of Form B.

### DETAILED DESCRIPTION

D-type crystal of a compound represented by formula (I) 4-(7-hydroxy-4-oxo-2-propan-2-ylquinazolin-3-yl) benzonitrile, i.e., crystalline Form D, by using Cu-Kα radiation, has an X-ray powder diffraction pattern comprising characteristic peaks at the 2θ values of 6.7°±0.2°, 10.5°±0.2°, 4.2°±0.2°,

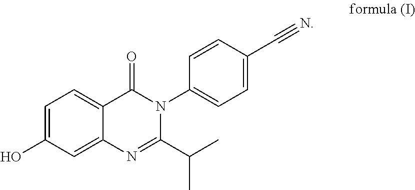 [SMILES: CC(C)c1nc2cc(O)ccc2c(=O)n1-c1ccc(C#N)cc1]

In an embodiment of the present disclosure, Form D has an X-ray powder diffraction pattern comprising characteristic peaks at one, two or three 2θ values of 21.0°±0.2°, 8.5°±0.2°, and 24.0°±0.2°.

In an embodiment of the present disclosure, Form D has an X-ray powder diffraction pattern comprising characteristic peaks at 2θ values of 21.0°±0.2°, 8.5°±0.2°, and 24.0°±0.2°.

In an embodiment of the present disclosure, Form D has an X-ray powder diffraction pattern comprising characteristic peaks at one, two or three 2θ values of 13.5°±0.2°, 27.3°±0.2°, and 14.6°±0.2°.

In an embodiment of the present disclosure, Form D has an X-ray powder diffraction pattern comprising characteristic peaks at 2θ values of 13.5°±0.2°, 27.3°±0.2°, and 14.6°±0.2°.

In an embodiment of the present disclosure, Form D has an X-ray powder diffraction pattern comprising characteristic peaks at four, or five, or six, or seven, or eight, or nine 2θ values of 6.7°±0.2°, 10.5°±0.2°, 4.2°±0.2°, 21.0°±0.2°, 8.5°±0.2°, 24.0°±0.2°, 13.5°±0.2°, 27.3°±0.2°, and 14.6°±0.2°.

In an embodiment of the present disclosure, Form D has an X-ray powder diffraction pattern comprising characteristic peaks at 2θ values of 6.7°±0.2°, 10.5°±0.2°, 4.2°±0.2°, 21.0°±0.2°, 8.5°±0.2°, 24.0°±0.2°, 13.5°±0.2°, 27.3°±0.2°, and 14.6°±0.2°.

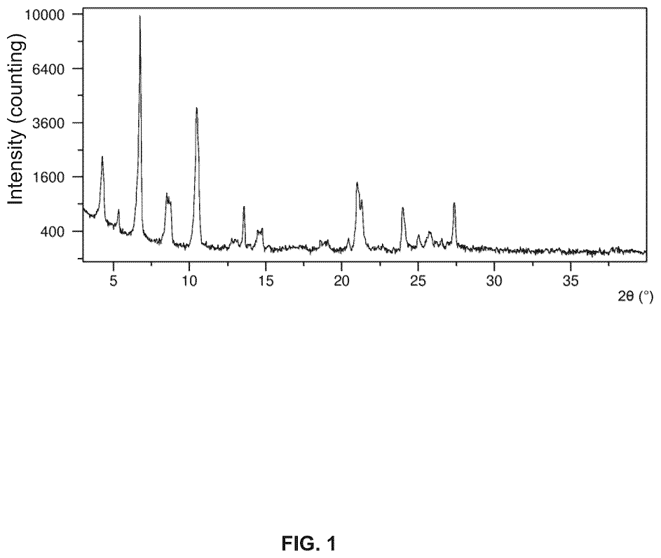

In an embodiment of the present disclosure, Form D has an X-ray powder diffraction pattern as shown in FIG. 1.

The process for preparing Form D includes steps (1) or (2):

(1) dissolving the compound shown in formula (I) into a solvent which includes a mixture of an alcohol solvent and pure water to obtain a solution, and then evaporating the solution until a solid is precipitated to obtain Form D.

According to the process for preparing Form D, the temperature for dissolving and evaporation is 20° C. to 30° C.

According to the process for preparing Form D, the alcohol solvent is methanol.
(2) dissolving the compound shown in formula (I) into alcohol solvent, and then add in polymers; with inducing by polymers, evaporating the solution until a solid is precipitated to obtain Form D.

According to the process for preparing Form D, the alcohol solvent is methanol.

According to the process for preparing Form D, the temperature for evaporation is 20° C. to 30° C.

According to the process for preparing Form D, the polymers include polyvinyl pyrrolidone (PVP), polyvinyl alcohol (PVA), polyvinylchloride (PVC), polyvinyl acetate (PVAC), hypromellose (HPMC), methyl cellulose (MC), polycaprolactone (PCL), polyethylene glycol (PEG), poly (methyl methacrylate) (PMMA) sodium alginate (SA) or hydroxyethyl cellulose (HEC).

AJ-type crystal of a compound represented by formula (I) 4-(7-hydroxy-4-oxo-2-propan-2-ylquinazolin-3-yl) benzonitrile, i.e., crystalline Form AJ, by using Cu-Kα radiation, has an X-ray powder diffraction pattern comprising characteristic peaks at the 2θ values of 24.5°±0.2°, 14.0°±0.2°, 10.5°±0.2°,

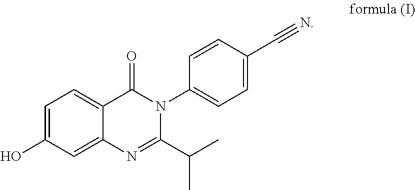 [SMILES: CC(C)c1nc2cc(O)ccc2c(=O)n1-c1ccc(C#N)cc1]

In an embodiment of the present disclosure, Form AJ has an X-ray powder diffraction pattern comprising characteristic peaks at one, two or three 2θ values of 16.4°±0.2°, 22.1°±0.2°, 19.0°±0.2°.

In an embodiment of the present disclosure, Form AJ has an X-ray powder diffraction pattern comprising characteristic peaks at 2θ values of 16.4°±0.2°, 22.1°±0.2°, 19.0°±0.2°.

In an embodiment of the present disclosure, Form AJ has an X-ray powder diffraction pattern comprising characteristic peaks at one, two or three 2θ values of 23.1°±0.2°, 12.0°±0.2°, 18.5°±0.2°.

In an embodiment of the present disclosure, Form AJ has an X-ray powder diffraction pattern comprising characteristic peaks at 2θ values of 23.1°±0.2°, 12.0°±0.2°, 18.5°±0.2°.

In an embodiment of the present disclosure, Form AJ has an X-ray powder diffraction pattern comprising characteristic peaks at four, or five, or six, or seven, or eight, or nine 2θ values of 24.5°±0.2°, 14.0°±0.2°, 10.5°±0.2°, 16.4°±0.2°, 22.1°±0.2°, 19.0°±0.2°, 23.1°±0.2°, 12.0°±0.2°, 18.5°±0.2°.

In an embodiment of the present disclosure, Form AJ has an X-ray powder diffraction pattern comprising characteristic peaks at 2θ values of 24.5°±0.2°, 14.0°±0.2°, 10.5°±0.2°, 16.4°±0.2°, 22.1°±0.2°, 19.0°±0.2°, 23.1°±0.2°, 12.0°±0.2°, 18.5°±0.2°.

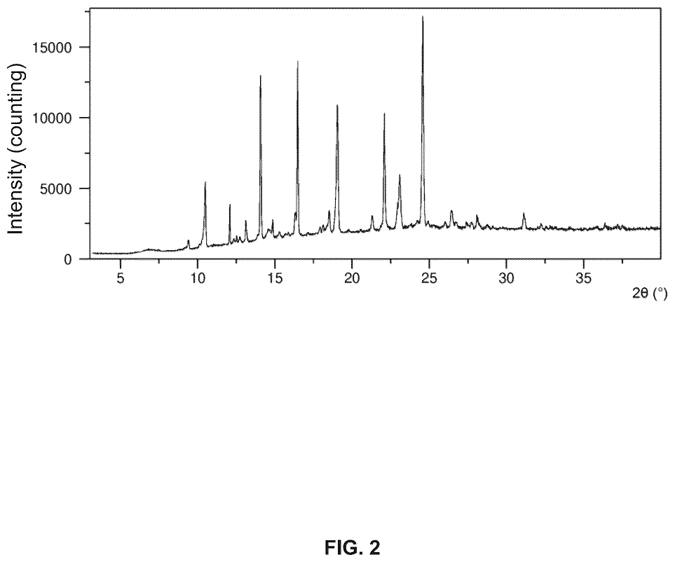

In an embodiment of the present disclosure, Form AJ has an X-ray powder diffraction pattern as shown in FIG. 2.

The process for preparing Form AJ includes
dissolving compound formula (I) in corresponding solvent, after filtration, dropwise adding anti-solvent into the solution to induce precipitates; and then heating the solid under N₂ flow at a certain temperature to obtain Form AJ. The corresponding solvent refers to methanol, acetone or tetrahydrofuran, and anti-solvent refers to water or n-heptane.

According to the process for preparing Form AJ, the heating temperature is 200° C. to 261° C., for example 253° C.

AL-type crystal of a compound represented by formula (I) 4-(7-hydroxy-4-oxo-2-propan-2-ylquinazolin-3-yl) benzonitrile, i.e., crystalline Form AL, by using Cu-Kα radiation, has an X-ray powder diffraction pattern comprising characteristic peaks at the 2θ values of 7.4°±0.2°, 14.9°±0.2°, 12.0°±0.2°

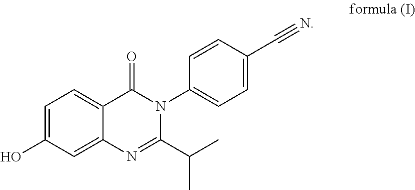 [SMILES: CC(C)c1nc2cc(O)ccc2c(=O)n1-c1ccc(C#N)cc1]

In an embodiment of the present disclosure, Form AL has an X-ray powder diffraction pattern comprising characteristic peak(s) at one, two, or three 2θ values of 19.2°±0.2°, 16.5°±0.2°, 17.8°±0.2°.

In an embodiment of the present disclosure, Form AL has an X-ray powder diffraction pattern comprising characteristic peak(s) at 2θ values of 19.2°±0.2°, 16.5°±0.2°, 17.8°±0.2°.

In an embodiment of the present disclosure, Form AL has an X-ray powder diffraction pattern comprising characteristic peak(s) at one, two, or three 2θ values of 11.2°±0.2°, 18.7°±0.2°, 28.4°±0.2°.

In an embodiment of the present disclosure, Form AL has an X-ray powder diffraction pattern comprising characteristic peak(s) at 2θ values of 11.2°±0.2°, 18.7°±0.2°, 28.4°±0.2°.

In an embodiment of the present disclosure, Form AL has an X-ray powder diffraction pattern comprising characteristic peaks at four, or five, or six, or seven, or eight, or nine 2θ values of 7.4°±0.2°, 11.2°±0.2°, 12.0°±0.2°, 14.9°±0.2°, 16.5°±0.2°, 17.8°±0.2°, 18.7°±0.2°, 19.2°±0.2°, 28.4°±0.2°.

In an embodiment of the present disclosure, Form AL has an X-ray powder diffraction pattern comprising characteristic peaks at 2θ values of 7.4°±0.2°, 11.2°±0.2°, 12.0°±0.2°, 14.9°±0.2°, 16.5°±0.2°, 17.8°±0.2°, 18.7°±0.2°, 19.2°±0.2°, 28.4°±0.2°.

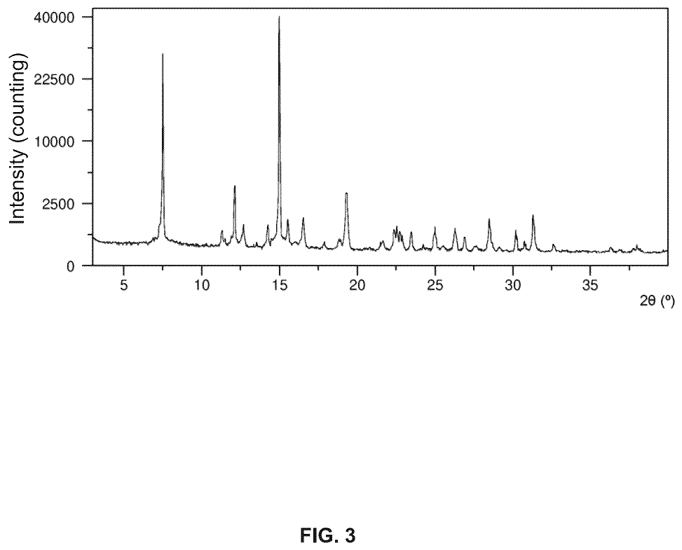

In an embodiment of the present disclosure, Form AL has an X-ray powder diffraction pattern as shown in FIG. 3.

The process for preparing Form AL includes:
(1) dissolving compound of formula (I) in ester solvent, and then evaporating the solution until precipitation; heating the obtained solid at 264° C. to obtain Form AL.

According to the process for preparing Form AL, the ester solvent is ethyl acetate.

According to the process for preparing Form AL, the temperature for dissolving and evaporation is 20° C. to 30° C.

According to the process for preparing Form AL, the heating rate is 5 -20° C./min, for example 10° C./min.

According to the process for preparing Form AL, after heating, the solid is cooled down to 20-40° C., for example 30° C.

(2) dissolving compound of formula (I) into cyclic ether solvent, and then evaporating the solution until precipitation to obtain Form AL.

According to the process for preparing Form AL, the cyclic ether solvent is cyclopentyl methyl ether.

According to the process for preparing Form AL, the temperature for dissolving and evaporation is 50° C. to 100° C., for example 80° C.

AZ-type crystal of a compound represented by formula (I) 4-(7-hydroxy-4-oxo-2-propan-2-ylquinazolin-3-yl) benzonitrile, i.e., crystalline Form AZ, by using Cu-Kα radiation, has an X-ray powder diffraction pattern comprising characteristic peaks at the 2θ values of 7.3°±0.2°, 7.9°±0.2°, 17.2°±0.2°,

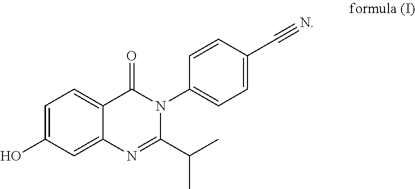 [SMILES: CC(C)c1nc2cc(O)ccc2c(=O)n1-c1ccc(C#N)cc1]

In an embodiment of the present disclosure, Form AZ has an X-ray powder diffraction pattern comprising characteristic peak(s) at one, two, or three 2θ values of 21.1°±0.2°, 22.6°±0.2°, 16.4°±0.2°.

In an embodiment of the present disclosure, Form AZ has an X-ray powder diffraction pattern comprising characteristic peak(s) at 2θ values of 21.1°±0.2°, 22.6°±0.2°, 16.4°±0.2°.

In an embodiment of the present disclosure, Form AZ has an X-ray powder diffraction pattern comprising characteristic peak(s) at one, two, or three 2θ values of 12.3°±0.2°, 26.6°±0.2°, 27.5°±0.2°.

In an embodiment of the present disclosure, Form AZ has an X-ray powder diffraction pattern comprising characteristic peak(s) at 2θ values of 12.3°±0.2°, 26.6°±0.2°, 27.5°±0.2°.

In an embodiment of the present disclosure, Form AZ has an X-ray powder diffraction pattern comprising characteristic peak(s) at four, or five, or six, or seven, or eight, or nine 2θ values of 7.3°±0.2°, 7.9°±0.2°, 17.2°±0.2°, 21.1°±0.2°, 22.6°±0.2°, 16.4°±0.2°, 12.3°±0.2°, 26.6°±0.2°, 27.5°±0.2°.

In an embodiment of the present disclosure, Form AZ has an X-ray powder diffraction pattern comprising characteristic peak(s) at 2θ values of 7.3°±0.2°, 7.9°±0.2°, 17.2°±0.2°, 21.1°±0.2°, 22.6°±0.2°, 16.4°±0.2°, 12.3°±0.2°, 26.6°±0.2°, 27.5°±0.2°.

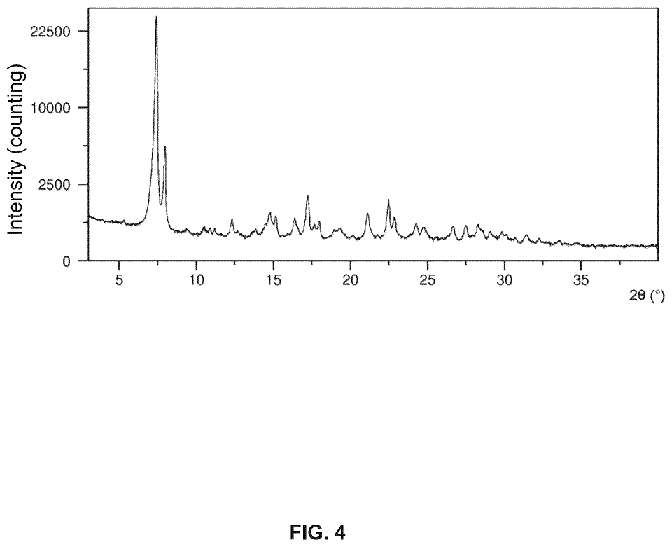

In an embodiment of the present disclosure, Form AZ has an X-ray powder diffraction pattern as shown in FIG. 4.

The process for preparing Form AZ includes:
dissolving compound of formula (I) in corresponding solvent, and then dropwise adding anti-solvent into the solution while stirring to induce precipitates; drying the solid at room temperature, and then keeping the solid for about 2 months to obtain Form AZ.

According to the process for preparing Form AZ, the corresponding solvent is ethyl acetate, and anti-solvent is n-heptane.

According to the process for preparing Form AZ, the temperature for precipitation and drying is 20° C. to 30° C.

According to the process for preparing Form AZ, the stirring temperature is −25° C. to 28° C., for example 25° C.

AF-type crystal of a compound represented by formula (I) 4-(7-hydroxy-4-oxo-2-propan-2-ylquinazolin-3-yl) benzonitrile, i.e., crystalline Form AF, by using Cu-Kα radiation, has an X-ray powder diffraction pattern comprising characteristic peaks at the 2θ values of 8.6°±0.2°, 10.0°±0.2°, 7.6°±0.2°,

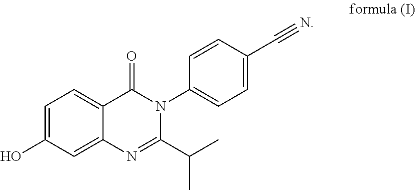 [SMILES: CC(C)c1nc2cc(O)ccc2c(=O)n1-c1ccc(C#N)cc1]

In an embodiment of the present disclosure, Form AF has an X-ray powder diffraction pattern comprising characteristic peak(s) at one, two, or three 2θ values of 17.3°±0.2°, 12.4°±0.2°, 28.3°±0.2°.

In an embodiment of the present disclosure, Form AF has an X-ray powder diffraction pattern comprising characteristic peak(s) at 2θ values of 17.3°±0.2°, 12.4°±0.2°, 28.3°±0.2°.

In an embodiment of the present disclosure, Form AF has an X-ray powder diffraction pattern comprising characteristic peak(s) at one, two, or three 2θ values of 22.2°±0.2°, 20.0°±0.2°, 14.8°±0.2°.

In an embodiment of the present disclosure, Form AF has an X-ray powder diffraction pattern comprising characteristic peak(s) at 2θ values of 22.2°±0.2°, 14.8°±0.2°.

In an embodiment of the present disclosure, Form AF has an X-ray powder diffraction pattern comprising characteristic peak(s) at four, or five, or six, or seven, or eight, or nine 2θ values of 8.6°±0.2°, 10.0°±0.2°, 7.6°±0.2°, 17.3°±0.2°, 12.4°±0.2°, 28.3°±0.2°, 22.2°±0.2°, 20.0°±0.2°, 14.8°±0.2°.

In an embodiment of the present disclosure, Form AF has an X-ray powder diffraction pattern comprising characteristic peak(s) at 2θ values of 8.6°±0.2°, 7.6°±0.2°, 17.3°±0.2°, 12.4°±0.2°, 28.3°±0.2°, 22.2°±0.2°, 20.0°±0.2°, 14.8°±0.2°.

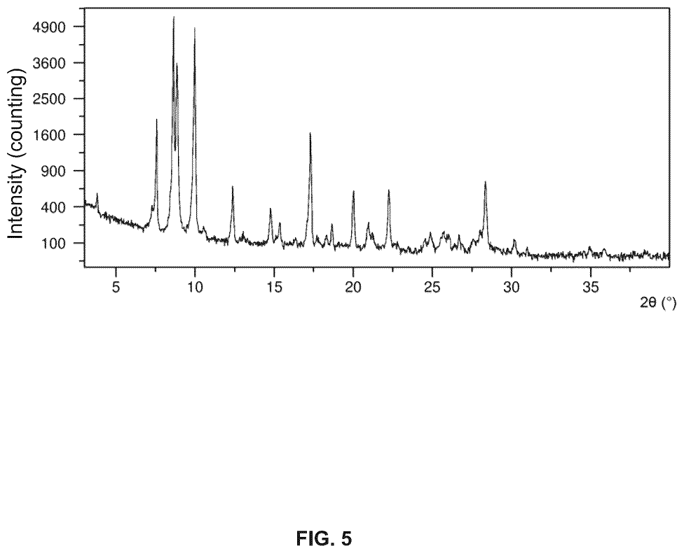

In an embodiment of the present disclosure, Form AF has an X-ray powder diffraction pattern as shown in FIG. 5.

The process for preparing Form AF includes:
dissolving compound of formula (I) in ketone solvent, and then evaporating the solution until a solid is precipitated; heating the obtained solid to a certain temperature, isothermal for some time, and then cooling back to room temperature to obtain Form AF.

According to the process for preparing Form AF, the ketone solvent is acetone.

According to the process for preparing Form AF, the temperature for dissolving and evaporation is 20° C. to 30° C.

According to the process for preparing Form AF, the heating temperature is 50° C. to 75° C., for example 55° C.

Z-type crystal of a compound represented by formula (I) 4-(7-hydroxy-4-oxo-2-propan-2-ylquinazolin-3-yl) benzonitrile, i.e., crystalline Form Z, by using Cu-Kα radiation, has an X-ray powder diffraction pattern comprising characteristic peaks at the 2θ values of 11.1°±0.2°, 18.5°±0.2°, 20.1°±0.2°,

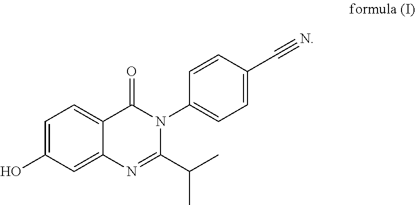 [SMILES: CC(C)c1nc2cc(O)ccc2c(=O)n1-c1ccc(C#N)cc1]

In an embodiment of the present disclosure, Form Z has an X-ray powder diffraction pattern comprising characteristic peak(s) at one, two, or three 2θ values of 12.1°±0.2°, 21.9°±0.2°, 19.1°±0.2°.

In an embodiment of the present disclosure, Form Z has an X-ray powder diffraction pattern comprising characteristic peak(s) at 2θ values of 12.1°±0.2°, 21.9°±0.2°, 19.1°±0.2°.

In an embodiment of the present disclosure, Form Z has an X-ray powder diffraction pattern comprising characteristic peak(s) at one, two, or three 2θ values of 23.3°±0.2°, 22.5°±0.2°, 26.5°±0.2°.

In an embodiment of the present disclosure, Form Z has an X-ray powder diffraction pattern comprising characteristic peak(s) at 2θ values of 23.3°±0.2°, 22.5°±0.2°, 26.5°±0.2°.

In an embodiment of the present disclosure, Form Z has an X-ray powder diffraction pattern comprising characteristic peak(s) at four, or five, or six, or seven, or eight, or nine 2θ values of 11.1°±0.2°, 18.5°±0.2°, 20.1°±0.2°, 12.1°±0.2°, 21.9°±0.2°, 19.1°±0.2°, 23.3°±0.2°, 22.5°±0.2°, 26.5°±0.2°.

In an embodiment of the present disclosure, Form Z has an X-ray powder diffraction pattern comprising characteristic peak(s) at 2θ values of 11.1°±0.2°, 18.5°±0.2°, 20.1°±0.2°, 12.1°±0.2°, 21.9°±0.2°, 19.1°±0.2°, 23.3°±0.2°, 22.5°±0.2°, 26.5°±0.2°.

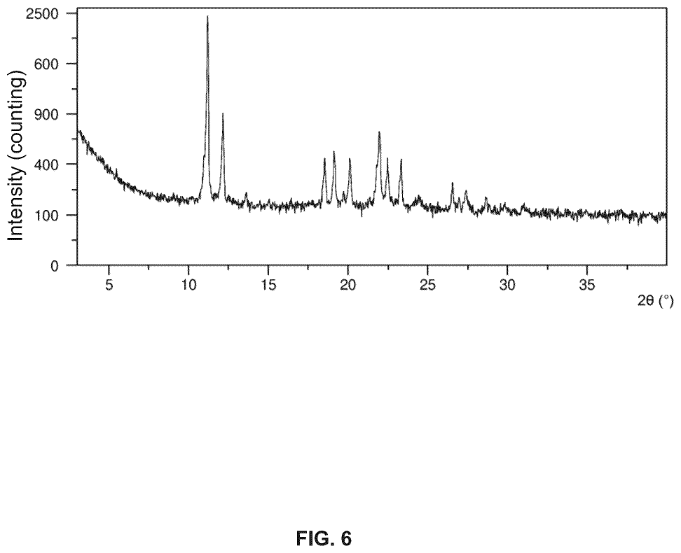

In an embodiment of the present disclosure, Form Z has an X-ray powder diffraction pattern as shown in FIG. 6.

The process for preparing Form Z includes:
(1) dissolving compound of formula (I) in ester solvent, after equilibrium at high temperature, cooling down the solution slowly until no precipitate is obtained; placing the solution under −20° C. for about 7 days, and then evaporating the solution at room temperature until a solid is precipitated to obtain Form Z.

According to the process for preparing Form Z, the ester solvent isopropyl acetate.

According to the process for preparing Form Z, the dissolving temperature is 40° C. to 60° C., for example 50° C.

According to the process for preparing Form Z, the solution is cooled down at a rate of 0.05° C./min to 0.5° C./min, for example 0.1° C./min.

According to the process for preparing Form Z, the evaporation temperature is 20° C. to 30° C.

(2) adding compound of formula (I) into organic solvent and equilibrating for some time to dissolve the solid, and then evaporating the solution at high temperature until a solid is precipitated to obtain Form Z. The organic solvent is methyl isobutyl ketone, isopropyl acetate or cyclopentyl methyl ether.

According to the process for preparing Form Z, the dissolving temperature is 40° C. to 80° C., for example 60° C.

According to the process for preparing Form Z, the evaporation temperature is 40° C. to 100° C., for example 80° C.

(3) dissolving compound of formula (I) in ester solvent, after equilibrium at high temperature, cooling down the solution quickly to a certain temperature, isothermal for some time and no precipitate is obtained; evaporating the solution at room temperature to obtain Form Z.

According to the process for preparing Form Z, the ester solvent is ethyl acetate.

According to the process for preparing Form Z, the dissolving temperature is 40° C. to 60° C., for example 50° C.

According to the process for preparing Form Z, the solution is cooled down to a target low temperature, for example −20° C.

According to the process for preparing Form Z, after cooling down, the solution is kept at low temperature for 1 to 12 days, for example 10 days.

According to the process for preparing Form Z, the evaporation temperature is 20° C. to 30° C.

(4) dissolving compound formula (I) in alcohol solvent, and then dropwise adding alkane solvent into the solution with stirring until precipitation to obtain Form Z.

According to the process for preparing Form Z, the alcohol solvent is ethanol.

According to the process for preparing Form Z, the alkane solvent is n-heptane.

According to the process for preparing Form Z, the temperature for dissolving and stirring is 20° C. to 30° C., for example 25° C.

AE-type crystal of a compound represented by formula (I) 4-(7-hydroxy-4-oxo-2-propan-2-ylquinazolin-3-yl) benzonitrile, i.e., crystalline Form AE, by using Cu-Kα radiation, has an X-ray powder diffraction pattern comprising characteristic peaks at the 2θ values of 12.6°±0.2°, 4.9°±0.2°, 14.0°±0.2°,

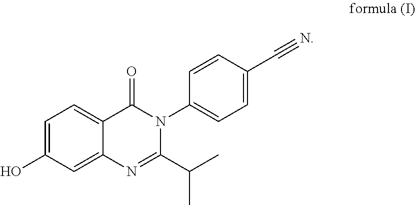 [SMILES: CC(C)c1nc2cc(O)ccc2c(=O)n1-c1ccc(C#N)cc1]

In an embodiment of the present disclosure, Form AE has an X-ray powder diffraction pattern comprising characteristic peak(s) at one, two, or three 2θ values of 11.4°±0.2°, 25.3°±0.2°.

In an embodiment of the present disclosure, Form AE has an X-ray powder diffraction pattern comprising characteristic peak(s) at 2θ values of 15.8°±0.2°, 11.4°±0.2°, 25.3°±0.2°.

In an embodiment of the present disclosure, Form AE has an X-ray powder diffraction pattern comprising characteristic peak(s) at one, two, or three 2θ values of 18.7°±0.2°, 23.7°±0.2°, 27.9°±0.2°.

In an embodiment of the present disclosure, Form AE has an X-ray powder diffraction pattern comprising characteristic peak(s) at 2θ values of 18.7°±0.2°, 23.7°±0.2°, 27.9°±0.2°.

In an embodiment of the present disclosure, Form AE has an X-ray powder diffraction pattern comprising characteristic peak(s) at four, or five, or six, or seven, or eight, or nine 2θ values of 12.6°±0.2°, 4.9°±0.2°, 14.0°±0.2°, 15.8°±0.2°, 11.4°±0.2°, 18.7°±0.2°, 23.7°±0.2°, 27.9°±0.2°.

In an embodiment of the present disclosure, Form AE has an X-ray powder diffraction pattern comprising characteristic peak(s) at 2θ values of 12.6°±0.2°, 4.9°±0.2°, 14.0°±0.2°, 15.8°±0.2°, 11.4°±0.2°, 25.3°±0.2°, 18.7°±0.2°, 23.7°±0.2°, 27.9°±0.2°.

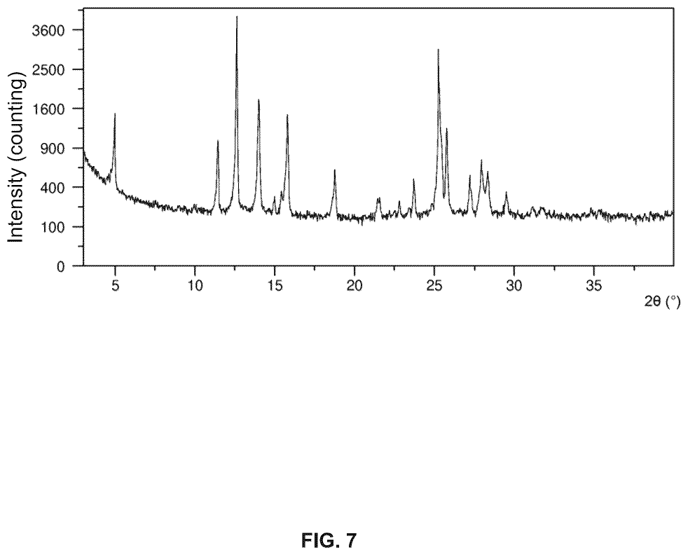

In an embodiment of the present disclosure, Form AE has an X-ray powder diffraction pattern as shown in FIG. 7.

The process for preparing Form AE includes:
dissolving compound of formula (I) in ester or ether solvent, and then evaporating the solution until precipitation to obtain Form AE.

According to the process for preparing Form AE, the ester solvent is isopropyl acetate.

According to the process for preparing Form AE, the ether solvent is cyclopentyl methyl ether.

According to the process for preparing Form AE, the evaporation temperature is 20° C. to 80° C.

According to the present disclosure, the compound of formula (I) and/or salt as raw material refers to its solid (crystal or amorphous), semi-solid, wax or oil form. As a preference, the compound of formula (I) and/or salt as raw material is in the form of solid powder. The “stirring” is completed by conventional methods, such as magnetic stirring or mechanical stirring, and the stirring speed is 50-1800 rpm, wherein the stirring speed for the magnetic stirring is 300-900 rpm, and the stirring speed for the mechanical stirring is 100-300 rpm.

In the present disclosure, “crystal”, “form”, “crystal form”, and/or “crystalline form” refers to a crystal or a crystalline form being identified by the X-ray diffraction pattern shown herein. Those skilled in the art are able to understand that physicochemical properties discussed herein can be characterized. The experimental errors depend on the instrument conditions, the sampling processes and the purity of samples. In particular, those skilled in the art generally know that the X-ray diffraction pattern typically varies with the experimental conditions. In particular, it is necessary to point out that, the relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and not used to absolute comparison. In addition, the experimental error of the diffraction peak position is usually 5% or less, and the error of these positions should also be taken into account. An error of ±0.2° is usually allowed. In addition, due to experimental factors such as sample thickness, the overall offset of the diffraction peak is caused, and a certain offset is usually allowed. Thus, it will be understood by those skilled in the art that a crystalline form of the present disclosure is not necessarily to have the exactly same X-ray diffraction pattern of the example shown herein and the “X-ray powder diffraction pattern is same” as described herein is not meaning absolutely the same, the same peak position can differ by ±0.2° and the peak intensity allows for some variability. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, crystalline forms of the present disclosure are pure, single, and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and further more specifically less than 1% (w/w).

It should be noted that a numerical value and a numerical range in the present disclosure should not be narrowly understood as the numerical value itself or the numerical range itself. It should be understood by those skilled in the art that the specific numerical value can be floated according to the specific technical environment on the basis of not departing from the spirit and principle of the present disclosure. In the present disclosure, the number of floating ranges which can be expected by one of skilled in the art is represented by the term “about”.

Crystalline Forms D, AJ, AL, AZ, AF, Z and AE of the compound of formula (I) provided by the present disclosure have the advantages in at least one of solubility, melting point, stability, dissolution, hygroscopicity, adhesion, fluidity, biological effectiveness, processing performance, purification, formulation production, safety, and the like, which provides a new and better choice for the preparation of pharmaceutical formulations containing formula (I), and has a very important significance for the drug development.

Compared with the prior art, crystalline forms of the compound of formula (I) provided by the present disclosure has the advantages in at least one of solubility, melting point, stability, dissolution, hygroscopicity, adhesion, fluidity, biological effectiveness, processing performance, purification, formulation production, safety, and the like, which provides a new and better choice for the preparation of pharmaceutical formulations of TRPV1 inhibitor, and has a very important significance for the drug development.

### EXAMPLES

The following will further illustrate the present disclosure through specific examples, which are not intended to limit the protection scope of the present disclosure. Those skilled in the art can make improvements to the preparation processes and the used instruments within the scope of the claims, and those improvements should be considered as falling into the protection scope of the present disclosure. Therefore, the protective scope of the present disclosure should be defined by the appended claims.

In the present disclosure, “room temperature” generally refers to 20° C. to 30° C., unless otherwise specified.

The abbreviations used in the present disclosure are explained as follows:
XRPD: X-ray Powder Diffraction
DSC: Differential Scanning calorimetry
TGA: ThermoGravimetric Analysis
UPLC: Ultra-high Performance Liquid Chromatography.
¹H NMR: Nuclear Magnetic Resonance Hydrogen spectrum

X-ray powder diffraction patterns of the present disclosure were collected on Empyrean-Type and X'Pert³ -Type X-ray powder diffractometers of PANalytacal Corporation. The process parameters of X-ray powder diffraction of the present disclosure were as follows:
X-ray source: Cu, Kα
Kα1 (Å): 1.54060; Kα2 (Å): 1.54443
Kα2/Kα1 intensity ratio: 0.50
Voltage: 45 kilovolts (kV)
Current: 40 milliamps (mA)
Scanning range: from 3.0 to 40.0 degrees (2θ angle).

Differential scanning calorimetry analysis charts of the present disclosure were collected on the Q200-type and Discovery DSC 2500-type differential scanning calorimeters of TA Company. The process parameters of the differential scanning calorimetry analysis of the present disclosure were as follows:
Scanning rate: 10° C./minute
Protection gas: Nitrogen gas.

The thermogravimetric analysis charts of the present disclosure were collected on Discovery TGA 5500-type and Q5000-type thermogravimetric analyzers of TA Company. The process parameters of the thermogravimetric analysis of the present disclosure were as follows:
Scanning rate: 10° C./minute
Protection gas: Nitrogen gas.

The UPLC data of the present disclosure were collected on Waters H Class, with photo-diode array detector. The UPLC parameters for purity and solubility test of the present disclosure were as follows:
1. Column: Waters Xbridge C18, 150×4.6 mm, 5 μm
2. Mobile phase: A: 0.05% trifluoroacetic acid in water; B: 0.05% trifluoroacetic acid in acetonitrile

Gradient ratio and time are listed in following table:

| Time (min) | % B |
|---|---|
| 0.0 | 5.0 |
| 3.0 | 40.0 |
| 3.5 | 90.0 |
| 3.8 | 90.0 |
| 3.9 | 5.0 |
| 5.0 | 5.0 |

3. Flow rate: 0.5 mL/min
4. Injection volume: 1 μL
5. Detector wavelength: 254 nm
6. Column temperature: 30° C.
7. Diluent: acetonitrile/water 1:1

The ion chromatograph (IC) data of the present disclosure were collected on ThermoFisher ICS-1100, the IC parameters for chloridion content test of the present disclosure were as follows:
1. Column: lonPac AS18 Analytical Column (4×250 mm)
2. Mobile phase: 25 mM aqueous sodium hydroxide solution
3. Flow rate: 1.0 mL/min
4. Injection volume: 25 μL
6. Column temperature: 35° C.
7. Cell temperature: 35° C.
8. Current: 80 mA
7. Run time: 7 minutes

The dynamic vapor sorption diagrams of the present disclosure were collected on Intrinsic-type and Intrinsic Plus-type dynamic vapor sorption instruments of SMS company. The process parameters of the dynamic vapor sorption test of the present disclosure were as follows:
Temperature: 25° C.
Protection gas and its flow rate: N₂, 200 mL/minute
dm/dt: 0.002%/minute
Minimum dm/dt equilibration time: 10 minutes
Maximum Equilibration Time: 180 minutes
Relative humidity range: 0% RH-95% RH-0% RH
Relative humidity gradient: 10% (0% RH-90% RH-0% RH), 5% (90% RH-95% RH and 95% RH-90% RH).

The particle size distribution results of the present disclosure were collected on S3500-type laser particle size analyzer of the Microtrac company. The Microtrac S3500 was equipped with an SDC (Sample Delivery Controller) sampling system. The wet method was used in this test, using Isopar G (containing 0.2% lecithin) as the test dispersion medium. The process parameters of the laser particle size analyzer were as follows.

| | |
|---|---|
| Particle size distribution: volume | Acquisition time: 10 seconds |
| distribution | |
| Dispersion medium: Isopar G | Particle size coordinate: Standard |
| Collection times: 3 times | Refractive index of dispersion medium: |
| | 1.42 |
| Transparency: Transparent | Residuals: Enabled |
| Particle refractive index: 1.59 | Flow rate: 60%* |
| Particle shape: Irregular | Filtration: Enabled |
| Ultrasonication power: 30 W | Ultrasonication time: 30 seconds |

*Flow rate 60% was 60% of 65 mL/s.

The intrinsic dissolution rate data of the present disclosure were collected on an Agilent 708DS-type dissolution apparatus of the Agilent company. The intrinsic dissolution test conditions were as follows.

| | |
|---|---|
| Dissolution apparatus | Agilent 708DS |
| Method | Paddle |
| Medium | pH 6.8 Phosphate Buffer |
| Medium volume | 900 mL |
| Rotation speed | 100 rpm |
| Medium temperature | 37° C. |
| Sampling points | 1, 2, 3, 4, 5, 10, 15, 20, 25, 30 minutes |
| Supplemental medium | No |

The polarized light microscope photos of the present disclosure were taken at room temperature with a Zeiss microscope Axio Scope.A1 equipped with an Axiocam 305 color camera and 5×, 10×, 20× and 50× objective lenses.

The compound represented by formula (I) which were used as freebase raw material in the following examples could be prepared according to the prior art, for example according to the process of WO2005120510A1. The solid form of raw material is not the key factor for preparation of the new crystalline forms of the present disclosure.

### Example 1: Preparation of Form D (Slow Evaporation)

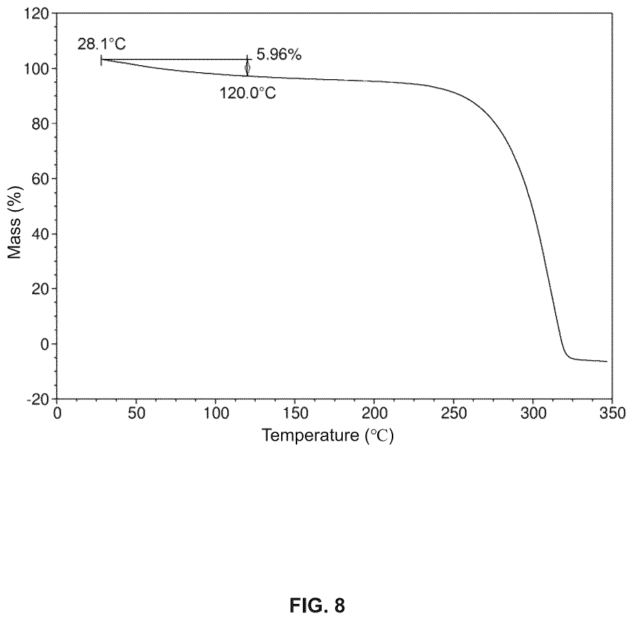

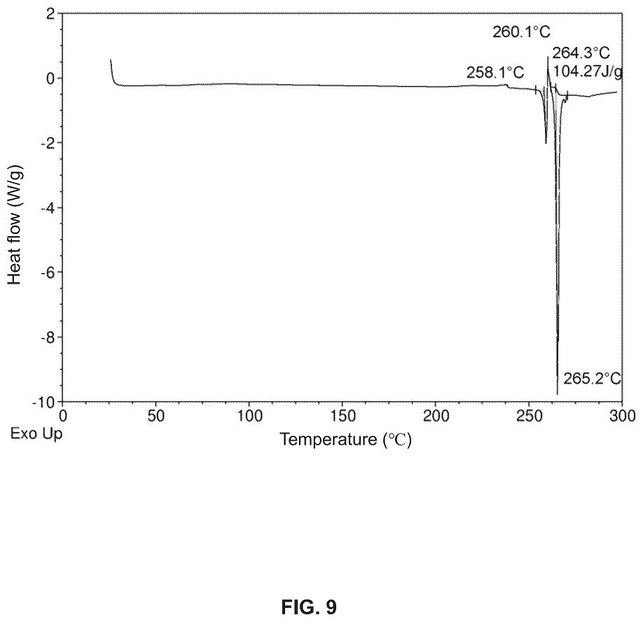

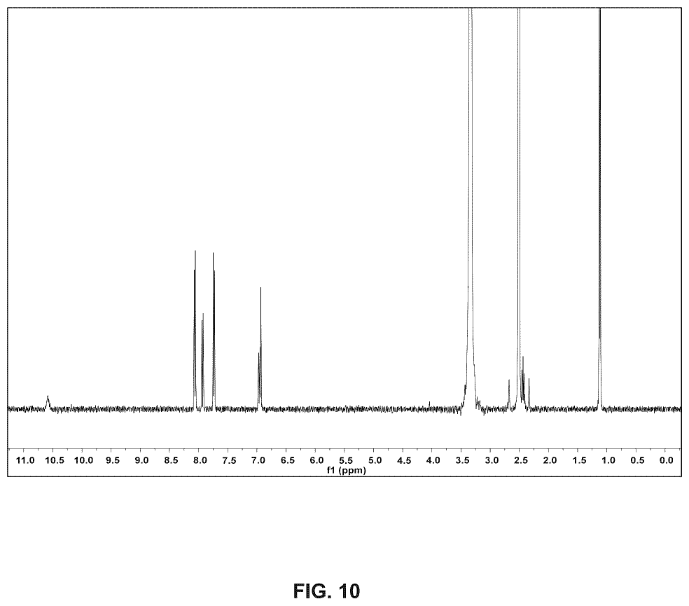

At room temperature, weigh 11.1 mg of solid compound represented by formula (I) into a 3-mL glass vial, and then add in 0.8 mL methanol to dissolve the solid. Filter the mixture into a new 3-mL glass vial using 0.45 μm PTFE filter membrane. Seal the vial with parafilm and then prick 4 pinholes. Evaporate the solution slowly at room temperature until precipitation to obtain Form D. The XRPD diagram of Form D was shown in FIG. 1, and the corresponding peak diffraction data was summarized in Table 1. TGA, DSC and ¹H NMR data were displayed as FIGS. 8 to 10.

TABLE 1

| Pos. [°2θ] | d-spacing [Å] | Rel. Int. [%] |
|---|---|---|
| 4.24 | 20.86 | 22.96 |
| 5.25 | 16.83 | 2.70 |
| 6.70 | 13.19 | 100.00 |
| 8.55 | 10.34 | 13.07 |
| 10.48 | 8.44 | 68.68 |
| 12.93 | 6.85 | 1.45 |
| 13.54 | 6.54 | 8.16 |
| 14.59 | 6.07 | 3.45 |
| 18.91 | 4.69 | 1.38 |
| 20.36 | 4.36 | 1.45 |
| 21.02 | 4.23 | 20.28 |
| 22.46 | 3.96 | 0.52 |
| 24.00 | 3.71 | 11.05 |
| 25.02 | 3.56 | 2.72 |
| 25.75 | 3.46 | 3.66 |
| 27.34 | 3.26 | 10.73 |
| 38.07 | 2.36 | 0.60 |

### Example 2: Preparation of Form D (Liquid Vapor Diffusion)

At room temperature, weigh 15.0 mg of solid compound re [resented by formula (I) into a 3-mL glass vial, and then add in 0.8 mL methanol to dissolve the solid. Filter the mixture into a new 3-mL glass vial using 0.45 μm PTFE filter membrane. Place the vial with open into a 20-mL glass vial with 4 mL of pure water. After sealing the vial and keeping the system at room temperature for liquid vapor diffusion for 8 days, solid was precipitated to obtain Form D. The corresponding peak diffraction data was summarized in Table 2.

TABLE 2

| Pos. [°2θ] | d-spacing [Å] | Rel. Int. [%] |
|---|---|---|
| 4.24 | 20.85 | 33.31 |
| 5.24 | 16.87 | 5.29 |
| 6.71 | 13.17 | 100.00 |
| 8.59 | 10.29 | 19.72 |
| 10.49 | 8.44 | 61.59 |
| 12.79 | 6.92 | 2.19 |
| 13.59 | 6.52 | 7.38 |
| 14.62 | 6.06 | 3.68 |
| 17.01 | 5.21 | 1.46 |
| 19.03 | 4.66 | 2.68 |
| 21.11 | 4.21 | 13.66 |
| 24.05 | 3.70 | 10.12 |
| 25.04 | 3.56 | 5.14 |
| 25.78 | 3.46 | 6.23 |
| 26.49 | 3.37 | 2.51 |
| 27.34 | 3.26 | 6.46 |
| 33.39 | 2.68 | 0.36 |
| 34.14 | 2.63 | 0.87 |

### Example 3: Preparation of Form AJ (Melting-Crystallization)

At room temperature, weigh 14.7 mg of compound represented by formula (I) solid in a 3-mL glass vial, and then add in 1.0 mL methanol to dissolve the solid. Filter the mixture into a new 20-mL vial using 0.45 μm PTFE filter membrane. With magnetically stirring (1000 rpm), dropwise add pure water into the filtrate to produce precipitate. Dry the solid at room temperature. Subsequently, weigh about 3˜5 mg of the obtained solid into XRPD sampler, and then heat the solid to 253° C. at a rate of 10° C./min under N₂ flow to obtain Form AJ. The XRPD diagram of Form AJ was shown in FIG. 2, and the corresponding peak diffraction data was summarized in Table 3.

TABLE 3

| Pos. [°2θ] | d-spacing [Å] | Rel. Int. [%] |
|---|---|---|
| 6.89 | 12.82 | 1.29 |
| 9.37 | 9.44 | 4.83 |
| 10.46 | 8.46 | 29.45 |
| 11.00 | 8.04 | 2.58 |
| 12.05 | 7.35 | 19.99 |
| 12.31 | 7.19 | 4.40 |
| 12.50 | 7.08 | 5.43 |
| 12.71 | 6.96 | 4.97 |
| 13.09 | 6.76 | 11.82 |
| 14.04 | 6.31 | 76.12 |
| 14.58 | 6.08 | 7.57 |
| 14.83 | 5.98 | 11.93 |
| 15.26 | 5.81 | 6.46 |
| 15.68 | 5.65 | 5.41 |
| 15.84 | 5.59 | 5.71 |
| 16.29 | 5.44 | 13.70 |
| 16.44 | 5.39 | 79.08 |
| 17.12 | 5.18 | 5.59 |
| 17.90 | 4.95 | 7.70 |
| 18.08 | 4.91 | 8.45 |
| 18.49 | 4.80 | 14.80 |
| 19.03 | 4.66 | 61.96 |
| 19.74 | 4.50 | 5.76 |
| 20.53 | 4.33 | 5.80 |
| 21.28 | 4.18 | 11.55 |
| 22.05 | 4.03 | 54.82 |
| 23.06 | 3.86 | 29.28 |
| 23.76 | 3.74 | 6.71 |
| 24.21 | 3.68 | 8.22 |
| 24.54 | 3.63 | 100.00 |
| 24.89 | 3.58 | 8.05 |
| 26.00 | 3.43 | 7.39 |
| 26.41 | 3.37 | 12.05 |
| 26.46 | 3.37 | 11.54 |
| 26.69 | 3.34 | 7.06 |
| 27.36 | 3.26 | 6.87 |
| 27.70 | 3.22 | 6.33 |
| 28.05 | 3.18 | 9.49 |
| 28.73 | 3.10 | 4.81 |
| 31.09 | 2.87 | 9.58 |
| 32.21 | 2.78 | 4.16 |
| 32.55 | 2.75 | 2.90 |
| 34.10 | 2.63 | 1.82 |
| 35.80 | 2.51 | 1.79 |
| 36.33 | 2.47 | 3.12 |
| 37.18 | 2.42 | 1.90 |
| 37.48 | 2.40 | 1.69 |

### Example 4: Preparation of Form AL (Fast Evaporation-Heating by DSC)

At room temperature, weigh 488.1 mg of solid compound represented by formula (I) into a 65-mL glass vial, and then add in 40 mL ethyl acetate to form a clear solution. Filter the mixture into a new 65-mL glass vial using 0.45 μm PTFE filter membrane. Evaporate the solution at room temperature until solid is precipitated.

At room temperature, weigh appropriate amount of the above solid into a DSC pan. Heat the solid to 264° C. at a rate of 10° C./min, and then cool back to 30° C. Form AL was obtained, and the corresponding peak diffraction data was summarized in Table 4.

TABLE 4

| Pos. [°2θ] | d-spacing [Å] | Rel. Int. [%] |
|---|---|---|
| 7.44 | 11.88 | 100.00 |
| 11.28 | 7.84 | 1.71 |
| 12.05 | 7.35 | 1.43 |
| 12.61 | 7.02 | 1.63 |
| 14.18 | 6.25 | 1.66 |
| 14.93 | 5.93 | 85.35 |
| 15.47 | 5.73 | 3.56 |
| 16.47 | 5.38 | 4.98 |
| 18.77 | 4.73 | 0.87 |
| 19.22 | 4.62 | 6.73 |
| 21.59 | 4.12 | 0.92 |
| 22.31 | 3.98 | 3.16 |
| 22.70 | 3.92 | 1.03 |
| 23.40 | 3.80 | 1.19 |
| 24.89 | 3.58 | 1.26 |
| 25.61 | 3.48 | 0.38 |
| 26.19 | 3.40 | 1.45 |
| 26.83 | 3.32 | 0.60 |
| 27.49 | 3.24 | 0.33 |
| 28.44 | 3.14 | 6.93 |
| 30.13 | 2.97 | 0.91 |
| 30.71 | 2.91 | 0.54 |
| 31.24 | 2.86 | 3.85 |
| 32.57 | 2.75 | 0.87 |
| 36.23 | 2.48 | 0.35 |

### Example 5: Preparation of Form AL (Fast Evaporation at High Temperature)

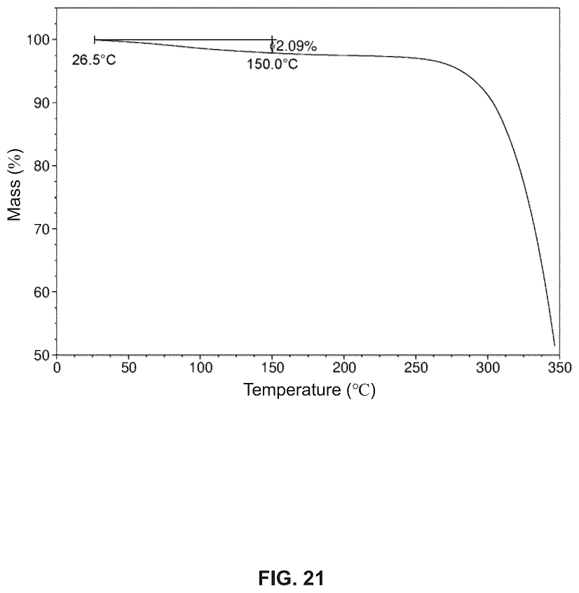

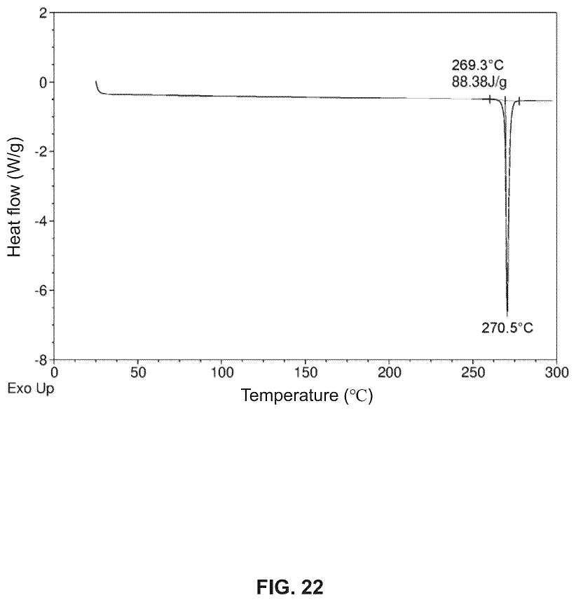

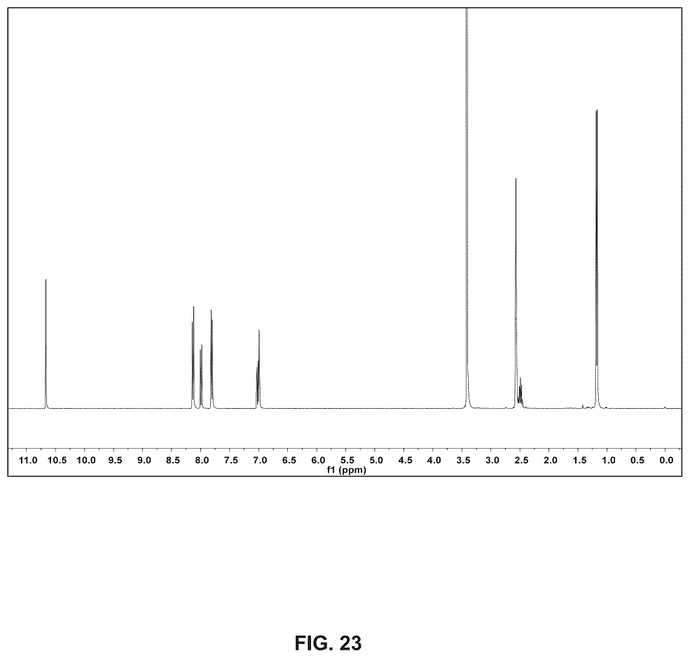

At room temperature, weigh 250.4 mg of compound formula (I) solid into a 65-mL glass vial, and then add in 35 mL cyclohexyl methyl ether to form a clear solution. Filter the mixture into a new 65-mL vial using 0.45 μm PTFE filter membrane. Evaporate the solution at 80° C. until precipitation to obtain Form AL. The XRPD diagram of Form AL was shown in FIG. 3 and the corresponding peak diffraction data was summarized in Table 5. TGA, DSC and ¹H NMR data were displayed as FIGS. 21 to 23.

TABLE 5

| Pos. [°2θ] | d-spacing [Å] | Rel. Int. [%] |
|---|---|---|
| 7.44 | 11.88 | 70.80 |
| 11.24 | 7.87 | 1.49 |
| 11.44 | 7.74 | 0.58 |
| 12.05 | 7.34 | 10.49 |
| 12.62 | 7.01 | 2.25 |
| 13.46 | 6.58 | 0.37 |
| 14.18 | 6.25 | 2.29 |
| 14.92 | 5.94 | 100.00 |
| 15.47 | 5.73 | 3.15 |
| 16.47 | 5.38 | 3.47 |
| 17.81 | 4.98 | 0.40 |
| 18.74 | 4.73 | 0.67 |
| 19.24 | 4.61 | 8.82 |
| 21.59 | 4.12 | 0.64 |
| 22.29 | 3.99 | 1.81 |
| 22.48 | 3.96 | 1.97 |
| 22.69 | 3.92 | 1.52 |
| 23.41 | 3.80 | 1.53 |
| 24.22 | 3.67 | 0.15 |
| 24.93 | 3.57 | 2.02 |
| 25.43 | 3.50 | 0.26 |
| 26.21 | 3.40 | 1.98 |
| 26.82 | 3.32 | 1.04 |
| 27.54 | 3.24 | 0.25 |
| 28.41 | 3.14 | 3.17 |
| 29.06 | 3.07 | 0.19 |
| 30.11 | 2.97 | 1.80 |
| 30.68 | 2.91 | 0.62 |
| 31.22 | 2.86 | 3.93 |
| 32.54 | 2.75 | 0.43 |
| 36.24 | 2.48 | 0.26 |
| 37.84 | 2.38 | 0.21 |

### Example 6: Preparation of Form AZ (Solid-state transformation)

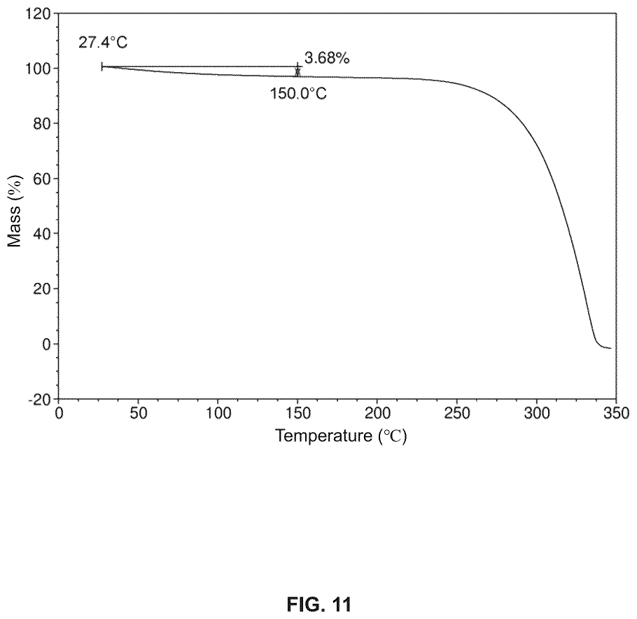

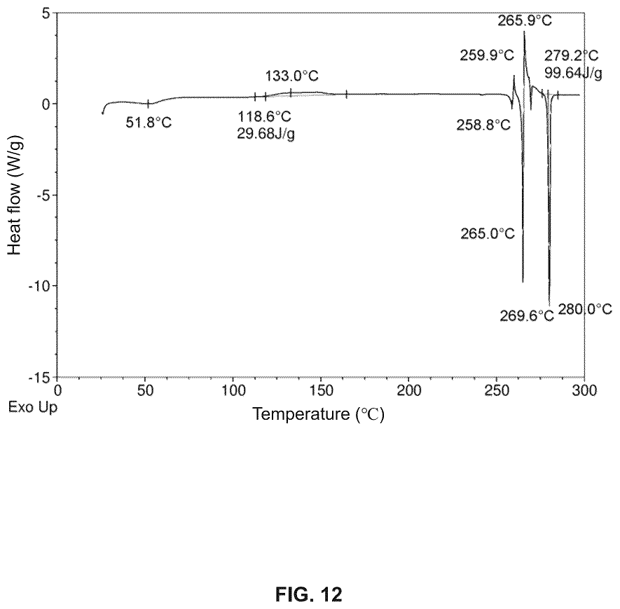

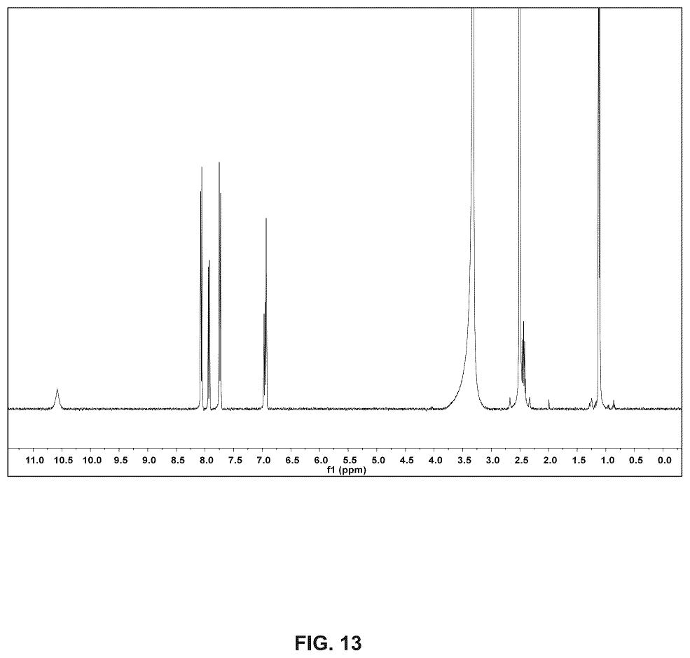

At room temperature, weigh 15.3 mg of compound represented by formula (I) into a 3-mL glass vial, and then add in 1.0 mL ethyl acetate to dissolve the solid. Filter the mixture into a new 20-mL vial using 0.45 μm PTFE filter membrane. With magnetically stirring (1000 rpm), dropwise add n-heptane into the filtrate. After the volume of n-heptane reaches 3 mL, solid is precipitated. Keep the obtained solid at room temperature for 2 months to obtain Form AZ. The XRPD diagram of Form AZ was shown in FIG. 4, and the corresponding peak diffraction data was summarized in Table 6. TGA, DSC and ¹H NMR data were displayed as FIGS. 11 to 13.

TABLE 6

| Pos. [°2θ] | d-spacing [Å] | Rel. Int. [%] |
|---|---|---|
| 7.32 | 12.08 | 100.00 |
| 7.94 | 11.12 | 18.55 |
| 9.31 | 9.49 | 0.38 |
| 10.52 | 8.41 | 0.74 |
| 12.28 | 7.21 | 1.93 |
| 13.75 | 6.44 | 0.62 |
| 14.73 | 6.01 | 3.49 |
| 15.12 | 5.86 | 2.50 |
| 16.39 | 5.41 | 2.27 |
| 17.17 | 5.16 | 6.93 |
| 17.81 | 4.98 | 1.24 |
| 19.18 | 4.63 | 1.02 |
| 21.10 | 4.21 | 3.59 |
| 22.56 | 3.94 | 3.48 |
| 24.72 | 3.60 | 1.52 |
| 26.61 | 3.35 | 1.64 |
| 27.46 | 3.25 | 1.76 |
| 28.33 | 3.15 | 1.67 |
| 29.88 | 2.99 | 0.81 |
| 31.39 | 2.85 | 0.83 |
| 33.48 | 2.68 | 0.27 |
| 34.65 | 2.59 | 0.12 |

### Example 7: Preparation of Form AF (Heating-Transformation)

At room temperature, weigh 15.0 mg of compound represented by formula (I) into a 3-mL glass vial, and then add in 0.4 mL acetone to dissolve the solid. Filter the mixture into a new 3-mL vial using 0.45 μm PTFE filter membrane. Seal the vial with parafilm and then prick 4 pinholes. Evaporate the solution slowly at room temperature for 7 days to produce precipitates.

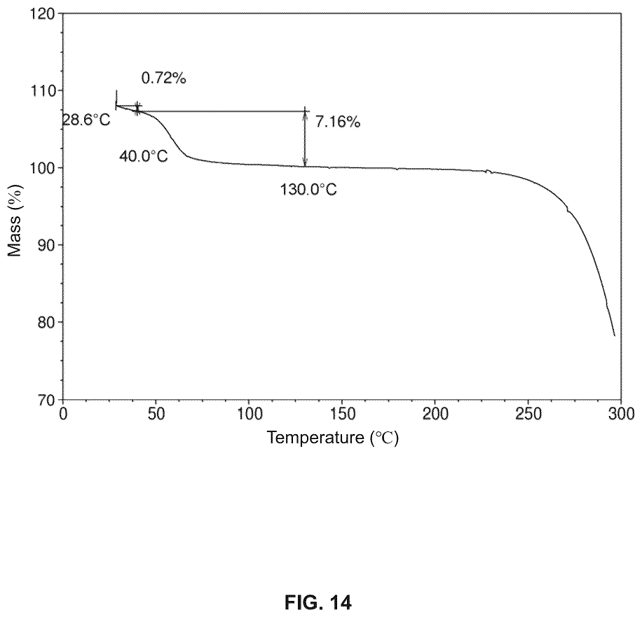

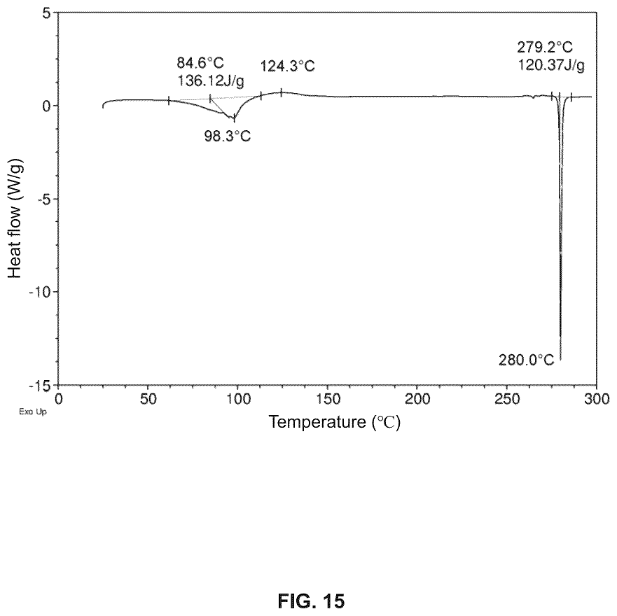

At room temperature, weigh appropriate amount of the above solid into a DSC pan. Ramp to 55° C. at a rate of 10° C./min, isothermal for 3 minutes and then cool back to 30° C. at a rate of 30° C./min. Form AF was obtained. The XRPD diagram of Form AF was shown in FIG. 5, and the corresponding peak diffraction data was summarized in Table 7. TGA and DSC data were displayed as FIGS. 14 to 15.

TABLE 7

| Pos. [°2θ] | d-spacing [Å] | Rel. Int. [%] |
|---|---|---|
| 3.79 | 23.30 | 5.70 |
| 7.25 | 12.19 | 4.14 |
| 7.55 | 11.71 | 31.30 |
| 8.62 | 10.26 | 100.00 |
| 8.85 | 10.00 | 64.95 |
| 9.96 | 8.88 | 87.62 |
| 10.54 | 8.39 | 1.43 |
| 12.37 | 7.16 | 10.57 |
| 13.01 | 6.80 | 0.79 |
| 14.76 | 6.00 | 5.64 |
| 15.35 | 5.77 | 3.10 |
| 16.32 | 5.43 | 0.96 |
| 17.28 | 5.13 | 29.78 |
| 17.65 | 5.02 | 1.20 |
| 18.29 | 4.85 | 1.27 |
| 18.64 | 4.76 | 3.06 |
| 19.99 | 4.44 | 9.79 |
| 20.94 | 4.24 | 3.30 |
| 21.21 | 4.19 | 1.90 |
| 22.23 | 4.00 | 10.35 |
| 24.51 | 3.63 | 1.08 |
| 24.87 | 3.58 | 1.91 |
| 25.68 | 3.47 | 2.20 |
| 26.00 | 3.43 | 1.64 |
| 26.40 | 3.37 | 0.80 |
| 26.67 | 3.34 | 1.48 |
| 27.57 | 3.23 | 1.50 |
| 27.93 | 3.19 | 2.66 |
| 28.34 | 3.15 | 13.10 |
| 30.17 | 2.96 | 1.40 |
| 34.94 | 2.57 | 0.61 |
| 35.82 | 2.51 | 0.50 |

### Examples 8-10: Preparation of Form Z (Fast Evaporation at High Temperature)

At room temperature, weigh appropriate amount of compound represented by formula (I) into a 3-mL glass vial, and then add in corresponding solvent to form a suspension. After being kept under 60° C. for about 1 hour, filter the mixture into a new 3-mL vial using 0.45 μm PTFE filter membrane. Evaporate the filtrate quickly at 80° C. until precipitation to obtain Form Z. The detailed procedure was listed in Table 8. The XRPD diagram of example 8 was shown in FIG. 6, and the corresponding peak diffraction data was summarized in Table 9.

TABLE 8

| | | Solvent | |
|---|---|---|---|
| Example | Mass (mg) | Type | Volume (mL) |
| 28 | 15.2 | Methyl isobutyl ketone | 0.4 |
| 29 | 15.1 | Isopropyl acetate | 0.6 |
| 30 | 15.0 | Cyclopentyl methyl ether | 0.6 |

TABLE 9

| Pos. [°2θ] | d-spacing [Å] | Rel. Int. [%] |
|---|---|---|
| 11.13 | 7.95 | 100.00 |
| 12.10 | 7.31 | 38.91 |
| 21.90 | 4.06 | 35.67 |
| 19.11 | 4.64 | 21.32 |
| 18.50 | 4.80 | 17.70 |
| 20.08 | 4.42 | 17.51 |
| 23.28 | 3.82 | 16.82 |
| 22.53 | 3.95 | 12.57 |
| 27.38 | 3.26 | 7.57 |
| 26.55 | 3.36 | 7.53 |
| 13.58 | 6.52 | 4.45 |
| 28.66 | 3.11 | 4.06 |
| 24.39 | 3.65 | 3.64 |
| 29.78 | 3.00 | 2.31 |
| 31.04 | 2.88 | 1.80 |

### Example 11: Preparation of Form Z (Slow Cooling-Fast Evaporation)

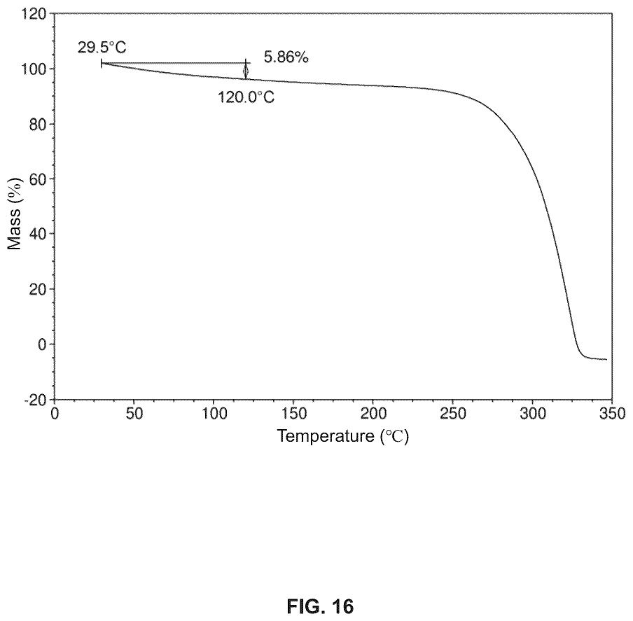

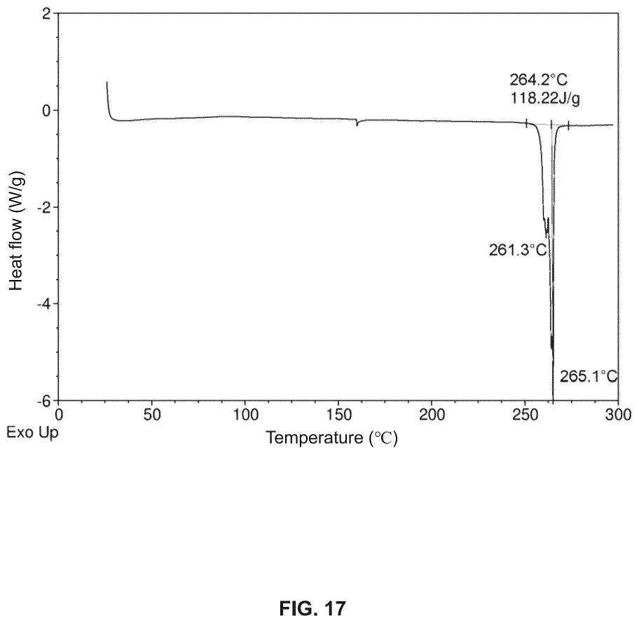

At room temperature, weigh 13.7 mg of compound represented by formula (I) into a 3-mL glass vial, and then add in 0.6 mL isopropyl acetate to form a suspension. After being kept under 50° C. for about 2 hours, filter the mixture into a new 3-mL vial using 0.45 μm PTFE filter membrane. Cap the filtrate and cool the system from 50° C. to 5° C. at a rate of 0.1° C./min, and then keep it under 5° C. for about 4 days. No solid was observed. Transfer the solution to −20° C. for about 6 days, and then no precipitate was observed. Evaporate the solution at room temperature until solid is precipitated to obtain Form Z, and the corresponding peak diffraction data was summarized in Table 10. TGA and DSC data were displayed as FIGS. 16 to 17.

TABLE 10

| Pos. [°2θ] | d-spacing [Å] | Rel. Int. [%] |
|---|---|---|
| 11.14 | 7.95 | 100.00 |
| 12.18 | 7.27 | 20.75 |
| 13.60 | 6.51 | 1.96 |
| 17.00 | 5.22 | 2.88 |
| 18.50 | 4.80 | 10.56 |
| 19.11 | 4.65 | 10.26 |
| 20.13 | 4.41 | 7.61 |
| 21.91 | 4.06 | 29.42 |
| 22.47 | 3.96 | 31.24 |
| 23.24 | 3.83 | 14.77 |
| 26.54 | 3.36 | 8.57 |
| 29.80 | 3.00 | 3.67 |

### Example 12: Preparation of Form Z (Crash Cooling-Fast Evaporation)

At room temperature, weigh 13.7 mg of compound represented by formula (I) in 3-mL glass vial, and then add in 0.4 mL 2,2,2-trifluoroethanol to form a suspension. After being kept under 50° C. for about 2 hours, filter the mixture into a new 3-mL vial using 0.45 μm PTFE filter membrane. Seal the vial and keep it under −20° C. for about 10 days. No solid was obtained. Evaporate the solution at room temperature until solid is precipitated to obtain Form Z, and the corresponding peak diffraction data was summarized in Table 11.

TABLE 11

| Pos. [°2θ] | d-spacing [Å] | Rel. Int. [%] |
|---|---|---|
| 11.15 | 7.94 | 100.00 |
| 12.12 | 7.31 | 28.40 |
| 12.27 | 7.21 | 20.47 |
| 13.57 | 6.53 | 2.07 |
| 14.95 | 5.93 | 0.49 |
| 15.37 | 5.77 | 0.39 |
| 17.45 | 5.08 | 0.34 |
| 18.48 | 4.80 | 3.76 |
| 19.08 | 4.65 | 4.97 |
| 19.68 | 4.51 | 3.82 |
| 20.05 | 4.43 | 3.50 |
| 21.74 | 4.09 | 10.83 |
| 21.93 | 4.05 | 39.83 |
| 22.46 | 3.96 | 46.23 |
| 22.93 | 3.88 | 2.70 |
| 23.27 | 3.82 | 14.51 |
| 24.41 | 3.65 | 1.39 |
| 26.50 | 3.36 | 3.21 |
| 26.93 | 3.31 | 0.97 |
| 27.35 | 3.26 | 3.31 |
| 28.61 | 3.12 | 1.34 |
| 29.14 | 3.07 | 0.64 |
| 29.78 | 3.00 | 0.90 |
| 31.01 | 2.88 | 0.26 |
| 33.03 | 2.71 | 0.87 |
| 33.91 | 2.64 | 0.64 |
| 34.72 | 2.58 | 1.08 |
| 35.45 | 2.53 | 1.22 |
| 38.87 | 2.32 | 0.41 |

### Example 13: Preparation of Form Z (Anti-Solvent Addition)

At room temperature, weigh 15.1 mg of compound represented by formula (I) in a 3-mL glass vial, and then add in 1.0 mL ethanol to dissolve the solid. Filter the mixture into a new 20-mL vial using 0.45 μm PTFE filter membrane. With magnetically stirring (1000 rpm), dropwise add 6 mL n-heptane into the solution to produce precipitates. Form Z was obtained and the corresponding peak diffraction data was summarized in Table 12.

TABLE 12

| Pos. [°2θ] | d-spacing [Å] | Rel. Int. [%] |
|---|---|---|
| 11.13 | 7.95 | 100.00 |
| 12.11 | 7.31 | 57.30 |
| 13.56 | 6.53 | 5.01 |
| 15.05 | 5.89 | 5.30 |
| 16.51 | 5.37 | 3.66 |
| 17.54 | 5.06 | 2.62 |
| 18.54 | 4.79 | 46.26 |
| 19.15 | 4.63 | 54.34 |
| 19.74 | 4.50 | 11.80 |
| 20.15 | 4.41 | 45.84 |
| 21.43 | 4.15 | 19.85 |
| 21.85 | 4.07 | 30.93 |
| 22.37 | 3.97 | 13.59 |
| 23.21 | 3.83 | 20.00 |
| 24.35 | 3.66 | 12.09 |
| 24.59 | 3.62 | 9.84 |
| 26.50 | 3.36 | 11.20 |
| 26.90 | 3.32 | 6.80 |
| 27.36 | 3.26 | 10.67 |
| 28.62 | 3.12 | 7.90 |
| 29.10 | 3.07 | 3.57 |
| 29.80 | 3.00 | 5.70 |
| 31.07 | 2.88 | 7.09 |
| 35.21 | 2.55 | 1.99 |
| 36.28 | 2.48 | 1.56 |
| 37.13 | 2.42 | 2.63 |
| 38.75 | 2.32 | 3.48 |
| 39.76 | 2.27 | 3.94 |

### Example 14: Preparation of Form AE (Fast Evaporation)

At room temperature, dissolve 10.0 mg of compound represented by formula (I) into 2.0 mL cyclopentyl methyl ether and sonicate to dissolve the solid. Filter the mixture into a new 3-mL vial using 0.45 μm PTFE filter membrane. Evaporate the filtrate at room temperature until solid is precipitated to obtain Form AE. The XRPD diagram of Form AE was shown in FIG. 7 and the corresponding peak diffraction data was summarized in Table 13.

TABLE 13

| Pos. [°2θ] | d-spacing [Å] | Rel. Int. [%] |
|---|---|---|
| 4.95 | 17.87 | 26.62 |
| 11.42 | 7.75 | 22.05 |
| 12.60 | 7.02 | 100.00 |
| 13.98 | 6.34 | 43.29 |
| 14.95 | 5.93 | 3.01 |
| 15.40 | 5.75 | 4.98 |
| 15.77 | 5.62 | 35.30 |
| 18.74 | 4.74 | 12.48 |
| 21.49 | 4.14 | 3.48 |
| 22.79 | 3.90 | 3.31 |
| 23.70 | 3.75 | 9.11 |
| 25.25 | 3.53 | 67.85 |
| 25.76 | 3.46 | 28.46 |
| 27.20 | 3.28 | 10.07 |
| 27.93 | 3.19 | 14.01 |
| 28.32 | 3.15 | 10.90 |
| 29.48 | 3.03 | 4.94 |

### Example 15: Preparation of Form AE (Slow Evaporation)

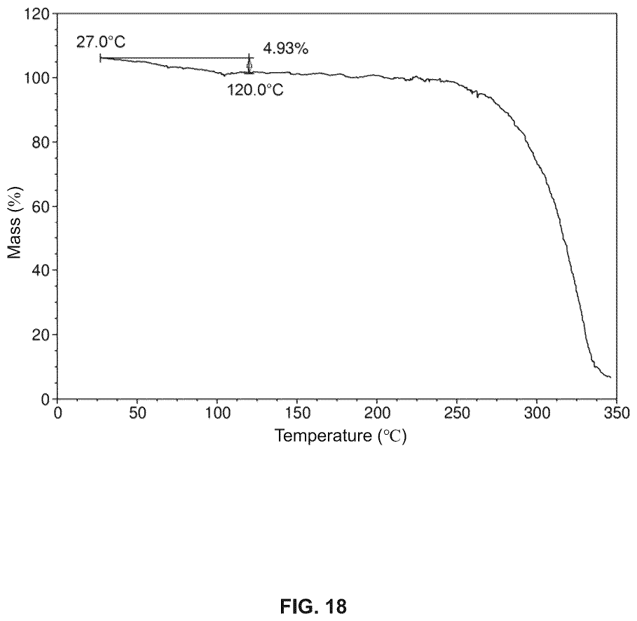

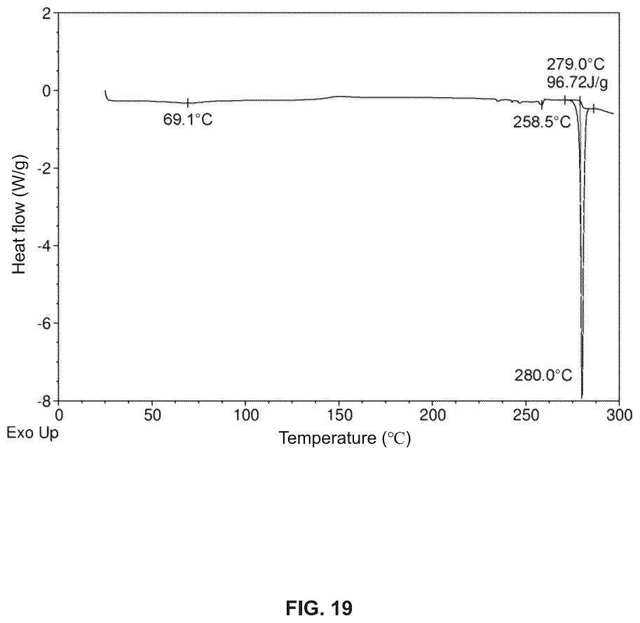

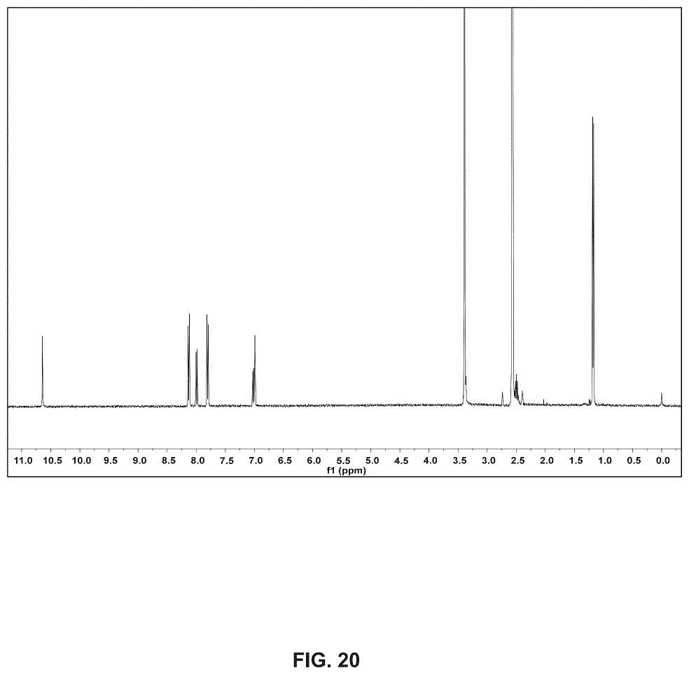

At room temperature, weigh 10.4 mg of compound formula (I) into a 3-mL glass vial, and then add in 2.0 mL isopropyl acetate to dissolve the solid. Filter the mixture into a new 3-mL vial using 0.45 μm PTFE filter membrane. Seal the vial and prick 4 pinholes. Evaporate the filtrate slowly at room temperature until precipitation to obtain Form AE. The corresponding peak diffraction data was summarized in Table 14. TGA, DSC and ¹H NMR data were displayed as FIGS. 18 to 20.

TABLE 14

| Pos. [°2θ] | d-spacing [Å] | Rel. Int. [%] |
|---|---|---|
| 4.95 | 17.87 | 28.49 |
| 6.48 | 13.63 | 0.26 |
| 9.92 | 8.91 | 0.87 |
| 11.35 | 7.80 | 0.80 |
| 12.14 | 7.29 | 0.75 |
| 12.57 | 7.04 | 100.00 |
| 13.95 | 6.35 | 0.54 |
| 14.93 | 5.93 | 1.96 |
| 15.37 | 5.76 | 0.33 |
| 15.71 | 5.64 | 0.62 |
| 18.70 | 4.75 | 0.22 |
| 23.63 | 3.76 | 0.48 |
| 25.04 | 3.56 | 2.31 |
| 25.36 | 3.51 | 17.13 |
| 27.98 | 3.19 | 0.50 |
| 31.20 | 2.87 | 0.07 |
| 35.33 | 2.54 | 0.09 |
| 36.54 | 2.46 | 0.06 |
| 38.41 | 2.34 | 0.72 |
| 38.50 | 2.34 | 0.92 |

### Example 16: Solubility of Crystalline Forms

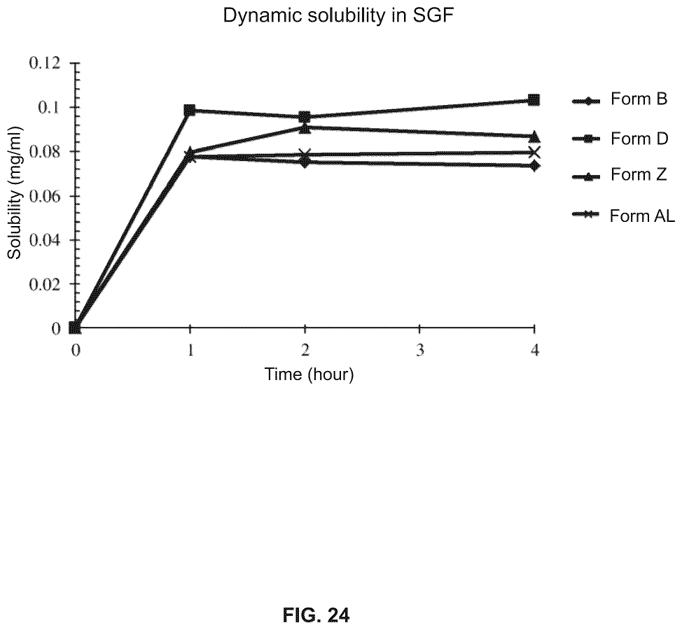

Suspend Forms D, AL, and Z of the present disclosure and Form B in the prior art in simulated gastric fluid (SGF) to form suspensions, respectively. After equilibrium for 1 hour, 2 hours and 4 hours, filter the suspensions to form saturated solutions. Determine the concentration of API in corresponding saturated solution by HPLC. The results were summarized in Table 15, and the solubility profile was shown in FIG. 24. As a result, Forms D, AL, and Z showed better solubility than Form B in SGF.

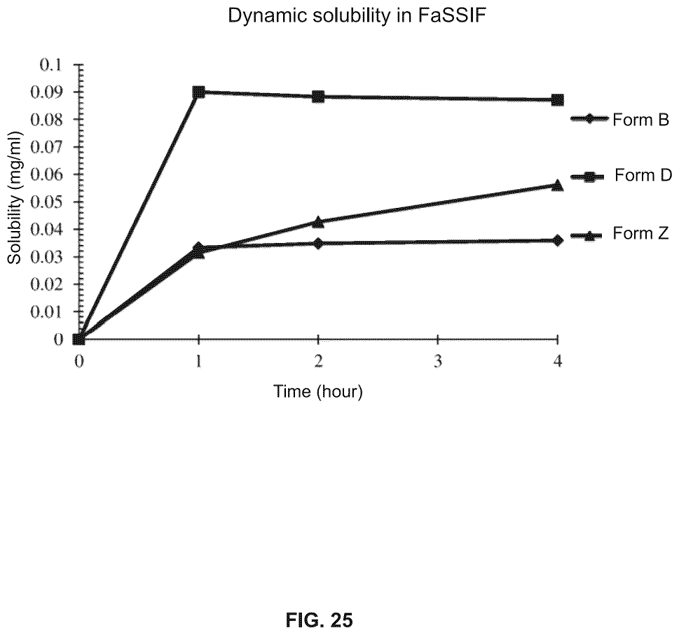

Suspend Forms D and Z of the present disclosure and Form B in the prior art in fasted-state simulated intestinal fluid (FaSSIF) to form suspensions, respectively. After equilibrium for 1 hour, 2 hours and 4 hours, filter the suspensions to form saturated solutions. Determine the concentration of API in corresponding saturated solution by HPLC. The results were summarized in Table 16, and the solubility profile was shown in FIG. 25. As a result, Forms D and Z showed better solubility than Form B in FaSSIF.

TABLE 15

| Bio-media | Time | Form B | Form D | Form AL | Form Z |
|---|---|---|---|---|---|
| SGF | 1 hr | 0.0775 | 0.0987 | 0.0774 | 0.0796 |
| (mg/mL) | 2 hr | 0.0752 | 0.0954 | 0.0788 | 0.0907 |
| | 4 hr | 0.0736 | 0.1033 | 0.0795 | 0.0867 |

TABLE 16

| Bio-media | Time | Form B | Form D | Form Z |
|---|---|---|---|---|
| FaSSIF | 1 hr | 0.0333 | 0.0900 | 0.0314 |
| (mg/mL) | 2 hr | 0.0348 | 0.0883 | 0.0427 |
| | 4 hr | 0.0359 | 0.0871 | 0.0561 |

### Example 17: Compressibility of Crystalline Forms

A manual tablet press was used for compression tableting. Appropriate amount of crystalline forms of the present disclosure and Form B in the prior art were compressed by circle punches under 10 kN pressure to form tablets. Hardness (H) of each tablet was determined by a tablet hardness tester after being kept in a dryer for 24 hours. Diameter (D) and thickness (L) of each tablet were determined by a vernier caliper, respectively. The tensile strength (T) of the tablets was calculated through the equation of T=2H/πDL. As a result, crystalline forms of the present disclosure showed a larger tensile strength than Form B and had a better compressibility.

### Example 18: Intrinsic Dissolution Rate of Crystalline Forms

Approximately 100 mg of crystalline forms of the present disclosure and Form B in the prior art were compressed by an intrinsic dissolution mold under 5 kN pressure to obtain thin tablets with each surface area of 0.5 cm², respectively. Instrinsic dissolution experiment was performed on the tablets, and the corresponding condition was shown in Table 17. The slope was calculated according to the value between 10 and 30 minutes (mg/mL), and the intrinsic dissolution rate (IDR) was further calculated according to the slope (mg/min/cm²). As a result, crystalline forms of the present disclosure showed a higher intrinsic dissolution rate than Form B.

TABLE 17

| Dissolution apparatus | CSE-051 Agilent 708DS | |
|---|---|---|
| Method | Paddle | |
| Media | pH = 6.8 phosphate buffer | |
| Media volume | 900 | mL |
| Stirring rate | 100 | rpm |
| Temperature | 37° | C. |

| | |
|---|---|
| Sampling time | 1, 2, 3, 4, 5, 10, 15, 20, 25, 30 mins |
| Media addition | No |

### Example 19: Solid-state Stability Study

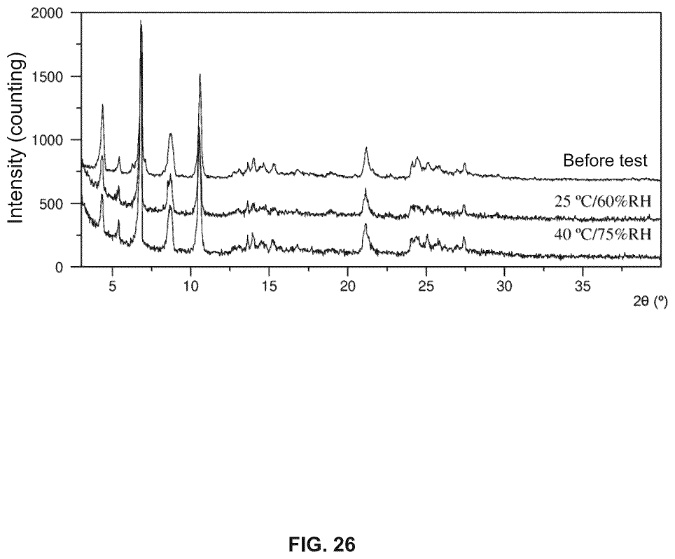

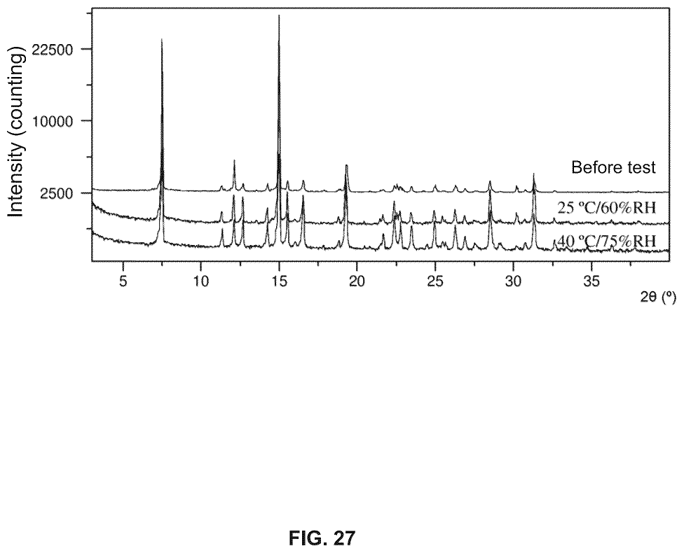

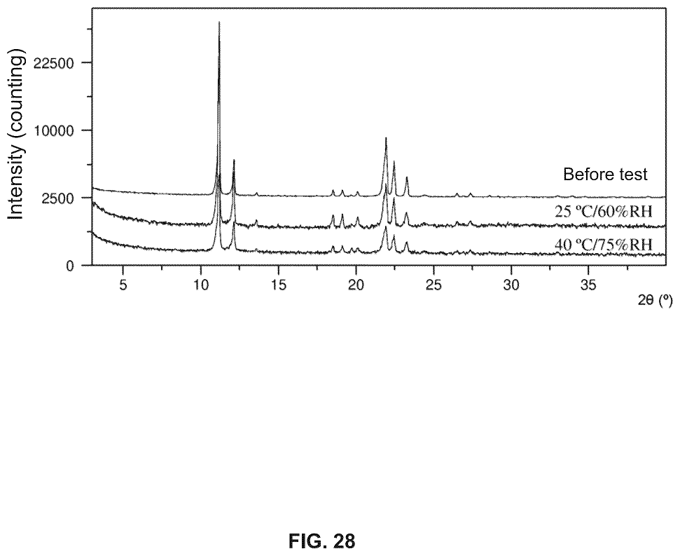

Weigh about 3 mg of Form D (initial purity: 99.59%), Form AL (initial purity: 100.00%) and Form Z (initial purity: 99.30%) of the present disclosure into vials, respectively. Place the vials under 25° C./60% RH and 40° C./75% RH for 7 days, and then separate several solids out for XRPD and HPLC purity test. The results were summarized in Table 18, and the XRPD overlay of Forms D, AL, and Z was shown in FIG. 26-28, respectively. As a result, Forms D, AL, and Z showed relatively good physical and chemical stability under 25° C./60% RH and 40° C./75% RH.

TABLE 18

| | | Solid form after | |
|---|---|---|---|
| Solid form | Condition | storage | Purity |
| Form D | 25° C./60% RH | Form D | 99.77% |
| Form AL | | Form AL | 100.00% |
| Form Z | | Form Z | 99.52% |
| Form D | 40° C./75% RH | Form D | 99.61% |
| Form AL | | Form AL | 100.00% |
| Form Z | | Form Z | 99.48% |

### Example 20: Hygroscopicity Study

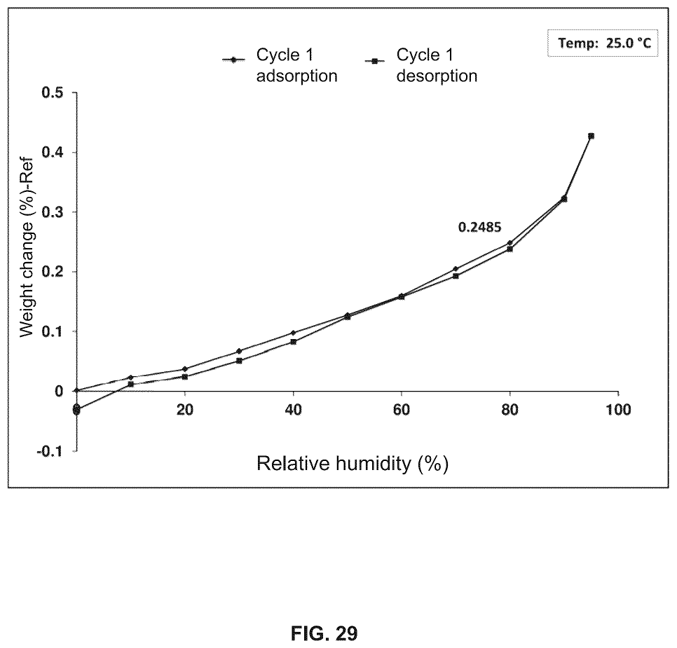

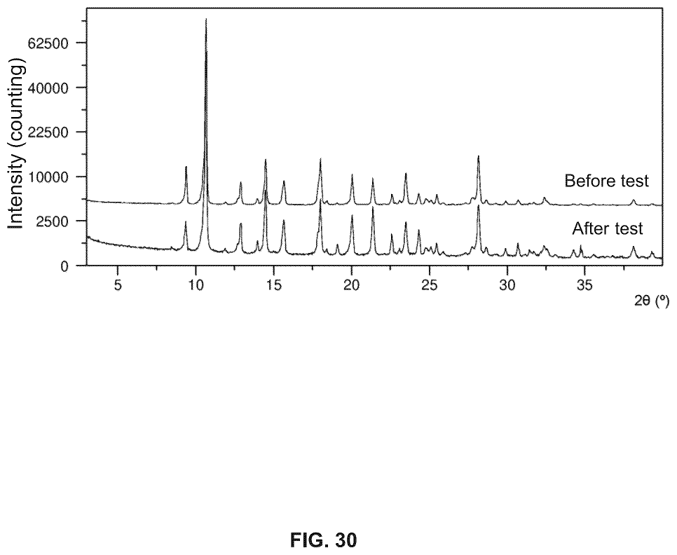

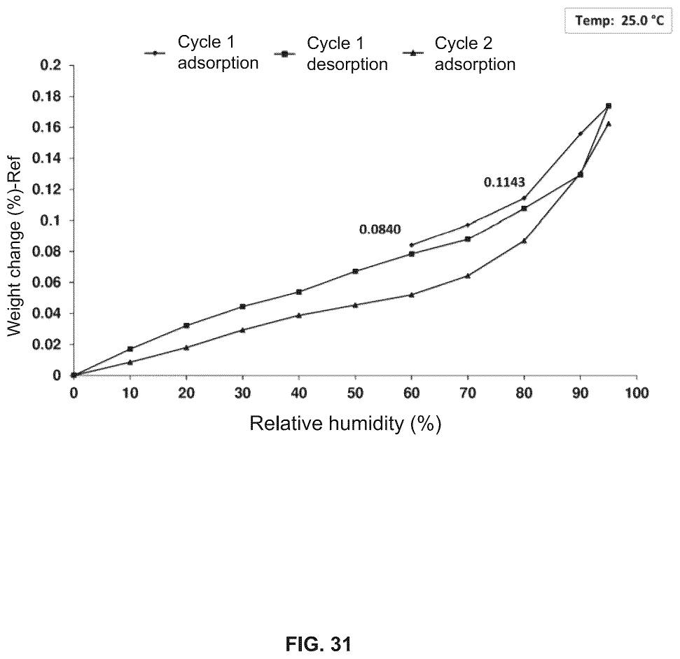

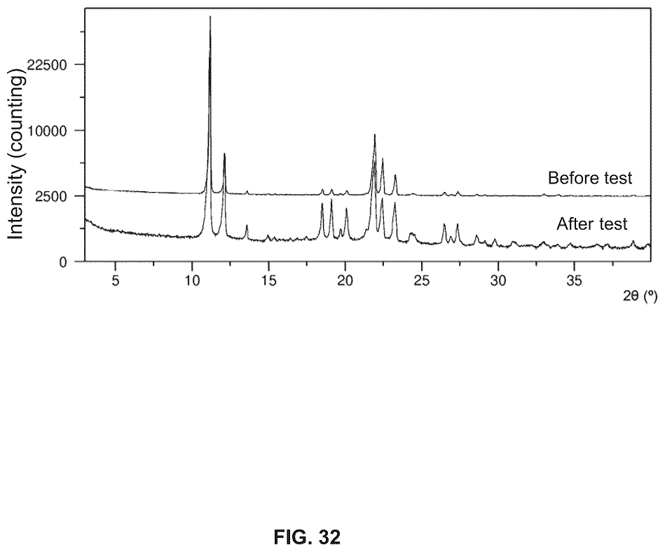

Weigh approximately 10 mg of Form Z of the present disclosure and Form B in the prior art for DVS test, and then collect the obtained solid for XRPD analysis. The results were summarized in Table 19, DVS plot of Form B was shown in FIG. 29, XRPD overlay of Form B was shown in FIG. 30, DVS plot of Form Z was shown in FIG. 31, and XRPD overlay of Form Z was shown in FIG. 32. As a result, Form Z showed relatively lower hygroscopicity than Form B.

TABLE 19

| Solid form | Water uptake at 80% RH | Hygroscopicity |
|---|---|---|
| Form B | 0.2485 | Slightly hygroscopic |
| Form Z | 0.0303 | Non-hygroscopic |

Description and definition of hygroscopicity (see appendix XIX J, guidance for hygroscopicity evaluation of Pharmacopoeia of the People's Republic of China 2010)
Deliquescent: sufficient water is absorbed to form a liquid,
Very hygroscopic: increase in mass is equal to or greater than 15 percent,
Hygroscopic: increase in mass is less than 15 percent and equal to or greater than 2 percent,
Slightly hygroscopic: increase in mass is less than 2 percent and equal to or greater than 0.2 percent.
Non-hygroscopic: increase in mass is less than 0.2 percent.

### Example 21: Crystal Habit Study

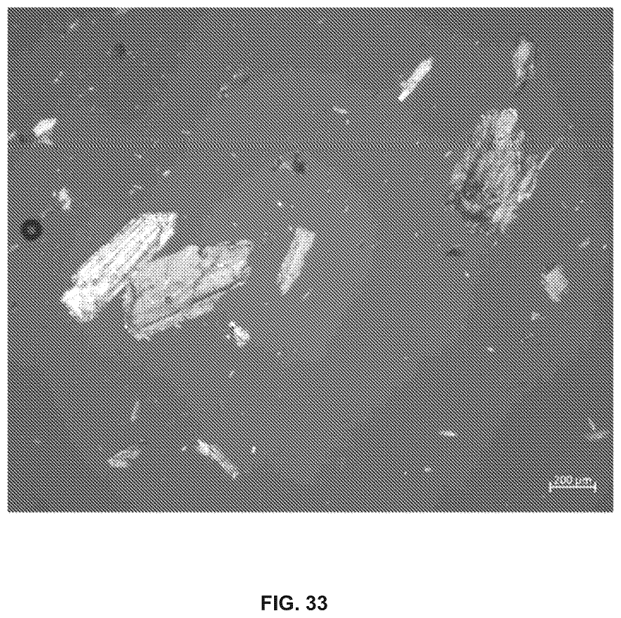

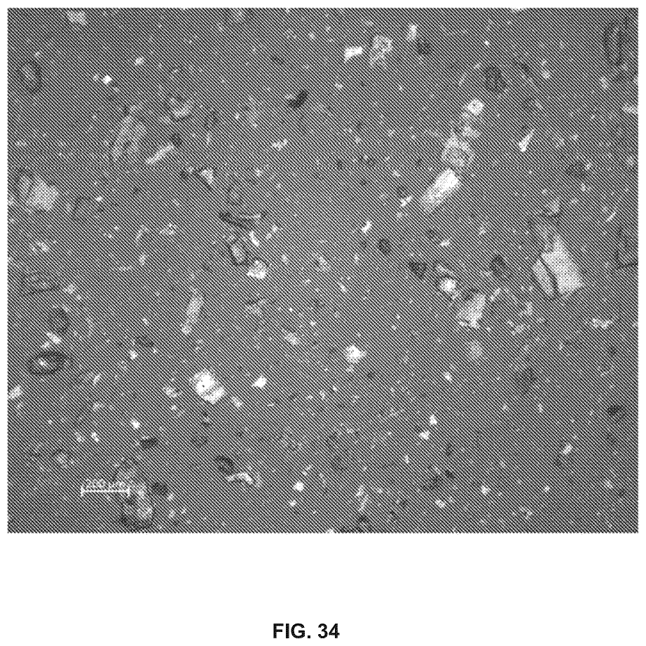

Weigh about 10 mg of Forms Z and AL of the present disclosure onto a glass slide, respectively. Disperse each solid with several vacuum pump oil, and then cover with a cover glass. Observe the sample using PLM. PLM images of Forms Z and AL were shown in FIGS. 33 to 34. As a result, Forms Z and AL samples were composed of regular particles, indicating Forms Z and AL have relatively good property on powder flow.

### Example 22: Particle Size Distribution Study

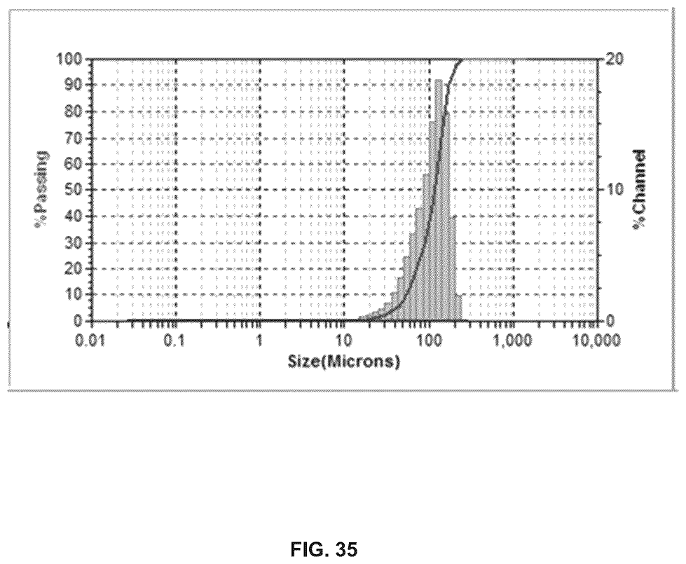

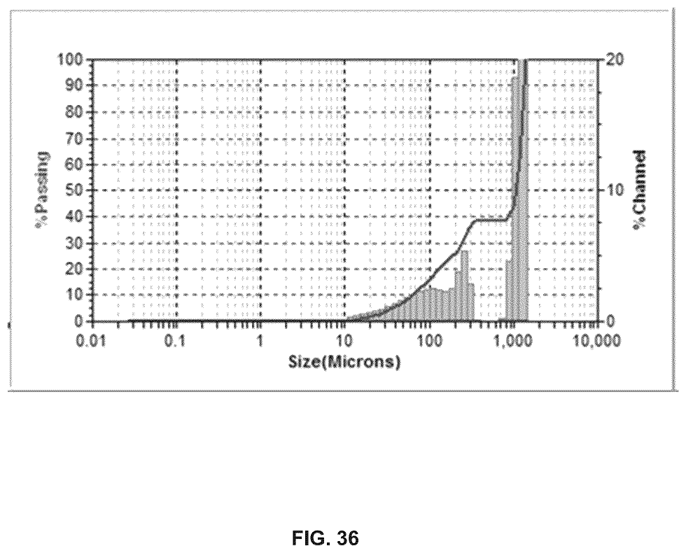

Approximately 10-30 mg of Form D of the present disclosure and Form B in the prior art were added into 5 mL Isopar G (0.2% lecithin) to form a suspension, respectively. The suspension was mixed well and then loaded into SDC loading system. Adjust the shading intensity to an appropriate range, and then started to collect data after sonication for 30 s. The results were summarized in Table 20, and the particle size distribution diagrams of Form D and Form B were shown in FIGS. 35 to 36, respectively. As a result, Form D of the present disclosure showed more uniform particle size distribution than Form B.

TABLE 20

| Solid form | MV (μm) | D10 (μm) | D50 (μm) | D90 (μm) |
|---|---|---|---|---|
| Form D | 116.2 | 53.9 | 117.2 | 175.9 |
| Form B | 795.2 | 62.3 | 1085.0 | 1331.0 |

### Example 23: Sticking Propensity

Approximately 30 mg of crystalline forms of the present disclosure and the prior form of Form B were weighed and then added into the dies of 8 mm round tooling, compressed at 10 kN pressure and held for 30 seconds. The punch was weighed and amount of material sticking to the punch was calculated. The compression was repeated once and the cumulative amount, maximum amount and average amount of material sticking to the punch during the compression were recorded. As a result, the adhesiveness of crystalline forms of the present disclosure is superior to Form B.

The above-mentioned embodiments are only intended to illustrate the technical concept and characteristics of the present disclosure, and the purpose thereof is to enable those who are familiar with the art to understand the content of the present disclosure and implement them accordingly, and cannot limit the protection scope of the present disclosure. All equivalent changes or modifications made according to the spirit of the present disclosure should be included within the protection scope of the present disclosure.

1. A crystalline Form D of a compound, represented by formula (I), with a chemical name of 4-(7-hydroxy-4-oxo-2-propan-2-ylquinazolin-3-yl) benzonitrile, wherein, by using Cu-Kα radiation, the crystalline Form D has an X-ray powder diffraction pattern comprising characteristic peaks at the 2θ values of 6.7°±0.2°, 4.2°±0.2°,

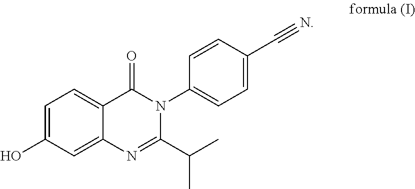 [SMILES: CC(C)c1nc2cc(O)ccc2c(=O)n1-c1ccc(C#N)cc1]

2. A process for preparing the crystalline Form D according to claim 1, comprising:
dissolving the compound represented by formula (I) into a solvent which includes a mixture of an alcohol solvent and pure water to obtain a solution, and evaporating the solution to obtain the crystalline Form D; or
dissolving the compound represented by formula (I) into an alcohol solvent, adding a polymer having 0.5% -15% mass of the compound represented by formula (I) to obtain a solution, the polymer selecting from polyvinyl pyrrolidone (PVP), polyvinyl alcohol (PVA), polyvinylchloride (PVC), polyvinyl acetate (PVAC), hypromellose (HPMC), methyl cellulose (MC), polycaprolactone (PCL.), polyethylene glycol (PEG), poly (methyl methacrylate) (PMMA), sodium alginate (SA) or hydroxyethyl cellulose (HEC); and evaporating the solution by induction of the polymer to obtain the crystalline Form D.

3-13. (canceled)

13. A crystalline Form AE of a compound, represented by formula (I), with a chemical name of 4-(7-hydroxy-4-oxo-2-propan-2-ylquinazolin-3-yl) benzonitrile, wherein, by using Cu-Kα radiation, the crystalline Form AE has an X-ray powder diffraction pattern comprising characteristic peaks at the 2θ values of 12.6°±0.2°, 4.9°±0.2°, 14.0°±0.2°,

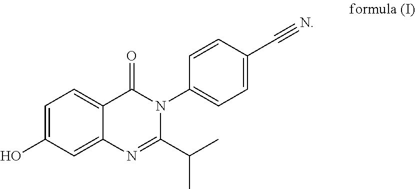 [SMILES: CC(C)c1nc2cc(O)ccc2c(=O)n1-c1ccc(C#N)cc1]

14. A process for preparing the crystalline Form AE according to claim 13, comprising:
dissolving compound represented by formula (I) in an ester or ether solvent to obtain a solution, and evaporating the solution to obtain the crystalline Form AE.

15-17. (canceled)

18. A crystalline Form of a compound, represented by formula (I), with a chemical name of 4-(7-hydroxy-4-oxo-2-propan-2-ylquinazolin-3-yl) benzonitrile, comprising crystalline Form AJ, crystalline Form AZ, or crystalline Form AF, wherein, by using Cu-Kα radiation,
the crystalline Form AJ has an X-ray powder diffraction pattern comprising characteristic peaks at the 2θ values of 24.5°±0.2°, 14.0°±0.2°, 10.5°±0.2°;
the crystalline Form AZ has an X-ray powder diffraction pattern comprising characteristic peaks at the 2θ values of 7.3°±0.2°, 7.9°±0.2°, 17.2°±0.2°; and
the crystalline Form AF has an X-ray powder diffraction pattern comprising characteristic peaks at the 2θ values of 8.6°±0.2°, 10.0°±0,2°, 7.6°±0.2°,

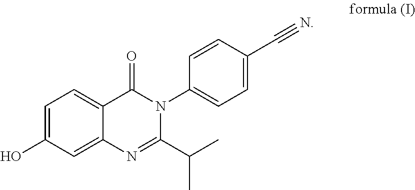 [SMILES: CC(C)c1nc2cc(O)ccc2c(=O)n1-c1ccc(C#N)cc1]

19. A process for preparing the crystalline Form AJ according to claim 18, comprising:
dissolving the compound represented by formula (I) in an alcohol, ketone or cyclic ether solvent to obtain a solution;
dropwise adding pure water or an alkane solvent into the solution until a solid is precipitated:
drying the solid at room temperature; and
heating the solid at 200-261° C. under N₂ flow to obtain the crystalline Form AJ.

20. A process for preparing the crystalline Form AZ according to claim 18, comprising:
dissolving the compound represented by formula (I) in an ester solvent;
dropwise adding an alkane solvent into a solution until a solid is precipitated; and
drying the solid at room temperature, and keeping the obtained solid for about 2 months to obtain the crystalline Form AZ.

21. A process for preparing the crystalline Form AF according to claim 18, comprising:
dissolving the compound represented by formula (I) in a ketone solvent to obtain a solution;
evaporating the solution until a solid is precipitated; and
heating the solid at 50° C.˜75° C., and cooling back to room temperature to obtain the crystalline Form AF.

22. A pharmaceutical composition, comprising the crystalline Form D according to claim 1 and a pharmaceutically acceptable carrier.

23. The pharmaceutical composition of claim 22, wherein the pharmaceutical composition has an inhibitory activity on transient receptor potential vanilloid 1 (TPRV1).

24. A method for preventing or treating pain, neurogenic bladder dysfunction, cancer pain, myofascial pain, inflammatory diseases, psoriasis, eczema, asthma, or pneumoconiosis, the method comprising: administering an effective amount of the crystalline Form D according to claim 1 to a subject.

25. A pharmaceutical composition, comprising the crystalline Form AE according to claim 13 and a pharmaceutically acceptable carrier.

26. The pharmaceutical composition of claim 28, wherein the pharmaceutical composition has an inhibitory activity on transient receptor potential vanilloid 1 (TPRV1).

27. A method for preventing or treating pain, neurogenic bladder dysfunction, cancer pain, myofascial pain, inflammatory diseases, psoriasis, eczema, asthma, or pneumoconiosis, the method comprising: administering an effective amount of the crystalline Form AE according to claim 13 to a subject.

28. A pharmaceutical composition, comprising the crystalline Form according to claim 18 and a pharmaceutically acceptable carrier.

29. The pharmaceutical composition of claim 28, wherein the pharmaceutical composition has an inhibitory activity on transient receptor potential vanilloid 1 (TPRV1).

30. A method for preventing or treating pain, neurogenic bladder dysfunction, cancer pain, myofascial pain, inflammatory diseases, psoriasis, eczema, asthma, or pneumoconiosis, the method comprising: administering an effective amount of the crystalline Form according to claim 18 to a subject.
